(19) United States
(12) Patent Application Publication
(10) Pub. No.: US 2015/0190080 A1
(43) Pub. Date: Jul. 9, 2015

(54) NEEDLE-MOUNTED LINEAR-ARRAY OXYGEN SENSOR

(71) Applicant: Regents of the University of Minnesota, Minneapolis, MN (US)

(72) Inventor: Shai Ashkenazi, St. Louis Park, MN (US)

(21) Appl. No.: 14/589,721

(22) Filed: Jan. 5, 2015

Related U.S. Application Data
(60) Provisional application No. 61/923,512, filed on Jan. 3, 2014.

(51) Int. Cl. A61B 5/1455; A61B 5/1459

(52) U.S. Cl. CPC A61B 5/14552; A61B 5/1459

(57) ABSTRACT

A low coherence light source is used to generate a time-varying and space-localized interference pattern to excite oxygen-sensitive dye embedded in a polymer matrix inside an elongated channel waveguide. The sensing mechanism may be based on triplet-state and phosphorescence quenching of the photosensitizer dye by oxygen molecules. Phosphorescence emission resulting from the time-varying, space-localized excitation light is collected. The intensity or frequency of an oscillating component of the phosphorescence signal is used to quantify the local value of pO₂ at a plurality of active measurement points along the waveguide. An oxygen sensor including the waveguide may be formed along a long axis of a needle so that a depth-resolved profile of pO₂ in tissue is obtained.

This application claims the benefit of U.S. Provisional Patent Application No. 61/923,512, filed Jan. 3, 2014, the entire contents of which are incorporated herein by reference.

### TECHNICAL FIELD

The disclosure relates to oxygen sensors.

### BACKGROUND

Oxygen sensors are used in a variety of applications, such as to measure oxygen levels within blood or tissue of a patient. The oxygen partial pressure (pO₂) in tissue is a parameter evaluated to assess tissue viability and cellular metabolism. Abnormally low levels of pO₂ may result from a variety of pathological conditions, such as diabetes, peripheral artery disease, immunologic and hematologic diseases that cause abnormalities in microcirculation, or hypercoagulable states. In some cases, lower limb blood flow at rest or with exercise may be associated with intermittent claudication or critical limb ischemia. For these diseases, example techniques and devices for measuring tissue oxygen may be imprecise, highly variable, present slow response times, or present technical difficulty to clinical sites and patients.

### SUMMARY

The disclosure is directed to techniques for measuring oxygen partial pressure at multiple points along a thin needle percutaneously inserted into a patient. Depth-resolved tissue oxygen profiles may be calculated based on the multiple points of measurement and displayed to a physician. In one example, an oxygen sensor may include a polymer-containing waveguide doped with an oxygen-sensing dye and a mirror placed at a distal end of the waveguide. The oxygen sensor may be disposed on or within a needle such that when the needle is inserted into tissue, oxygen present in the tissue diffuses into the waveguide and interacts with the oxygen-sensing dye.

In some example implementations, a low coherence light source (e.g., a continuous wave laser) may be optically coupled to the waveguide. Light from the light source may then be split into two waves and controlled to interfere along respective points (e.g., positions or sections) of the waveguide. Characteristic features of signals emitted by the excited oxygen-sensitive dye at each of the points along the needle, in response to an interference pattern generated by the interfering waves, may enable determination of pO₂ at different tissue depths corresponding to the points. Example needles including oxygen sensors, oxygen sensors including one or more optical waveguides, systems for implementing techniques of measuring pO₂ using oxygen sensors, and techniques including the referenced features (among others) are described in greater detail below.

In some example implementations, a depth-resolved pO₂ profile may be determined by measuring optical transient absorption of an oxygen-sensing dye along each of a plurality of sections of a dye-stained, polymer waveguide formed on a needle, following a pulse excitation of the respective section. In addition, optical absorption (and pO₂) of the multiple sections can be measured simultaneously. Some implementations may make use of a single fiber-optic cable for multi-point measurement by using a low coherence interferometry (LCI) technique. Other implementations may utilize multiple fiber-optic cables.

The disclosed techniques allow for the design of a very fine needle carrying a micro-waveguide structure capable of providing a profile of pO₂ along a long axis of the needle. For example, a micro-waveguide structure may allow for use of a small needle diameter (e.g., 0.15 millimeters (mm)), which may minimize both damage to blood vessels and patient pain levels. The small size of the oxygen sensor utilized also may facilitate a short response time (e.g., shorter than 1 second in example implementations).

In addition, an array of depth-resolved pO₂ values may provide a physician with enhanced diagnostic information, as compared to a sensor that measures a single point. In some examples of this disclosure, a needle may include a shaft and an oxygen sensor that includes an optical waveguide formed along the shaft, wherein the optical waveguide includes an outer core comprising a first polymer and an inner core comprising a second polymer containing an oxygen-sensing dye. The oxygen sensor also may include an optical coupler embedded within the shaft at a distal end of the waveguide to reflect light back through the waveguide.

In some examples, a system may include an oxygen sensor that includes an optical waveguide, where the optical waveguide includes an outer core and an inner core, the outer core comprising a first polymer and the inner core comprising a second polymer containing an oxygen-sensing dye. The oxygen sensor also may include an optical coupler disposed at a distal end of the waveguide to reflect light back through the waveguide. Further, the system may also include a controller comprising a light source, a processor, and a detector, where the light source is configured to be optically coupled to the waveguide of the oxygen sensor and deliver excitation light to the oxygen-sensing dye, where the processor is configured to control the excitation light to cause an interference pattern at multiple sections along the waveguide, and where the detector is configured to receive, via at least the waveguide, signals emitted by dye molecules of the oxygen-sensing dye in response to the interference pattern.

In some examples, a method for measuring oxygen partial pressure (pO₂) at multiple points along a needle may include delivering excitation light to an oxygen-sensing dye within an optical waveguide formed along a shaft of the needle; receiving, via at least the optical waveguide, signals emitted by dye molecules within the oxygen-sensing dye; calculating, based on the emitted signals, the phosphorescence intensity at multiple sections along the optical waveguide; and computing respective pO₂ values for each section based at least on the respective phosphorescence intensity at each section.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a conceptual diagram illustrating an example system including a needle-mounted oxygen sensor for measuring the oxygen partial pressure (pO₂) profile of tissue of a patient.

FIG. 2A is a conceptual diagram illustrating an axial cross-section of an example needle including an oxygen sensor.

FIG. 2B is a conceptual diagram of a radial cross-section of the needle of FIG. 2A along line A.

FIG. 3 is a schematic diagram illustrating an example system for measuring a depth-resolved pO₂ profile.

FIG. 4 is a chart illustrating a simulated result of interference zone emission produced by the system of FIG. 3.

FIG. 5 is a chart illustrating experimental results showing a square-wave phase modulation and a phosphorescence signal double-frequency response.

FIG. 6 is a flow diagram illustrating an example pO₂ measurement technique.

FIG. 7A is a conceptual diagram illustrating an axial cross-section of an example needle including an oxygen sensor with two optical waveguides.

FIG. 7B is a conceptual diagram illustrating a radial cross-section of the needle of FIG. 7A along line B.

FIG. 8 is a conceptual diagram illustrating a radial cross-section of another example needle including an oxygen sensor with two waveguides.

FIG. 9A is a top view of a conceptual diagram of an axial cross-section of another example needle including an oxygen sensor with two waveguides in a side-by-side arrangement.

FIG. 9B is a radial cross-section of the example needle of FIG. 9A along line C.

FIG. 10 is a schematic diagram illustrating another example system for measuring a depth-resolved pO₂ profile.

FIG. 11 is a conceptual diagram of a radial cross-sectional view of an example linear-array oxygen sensor including a double-cladding waveguide disposed on a needle.

FIGS. 12A and 12B illustrate two example designs of an inner core of a linear array oxygen sensor including a waveguide.

FIG. 13 is a graph illustrating an example measurement of transient optical absorption of a dye following excitation.

FIG. 14 is a schematic diagram illustrating an LCI-based system for measuring light transmission along the waveguide of an oxygen sensor at multiple points.

### DETAILED DESCRIPTION

The disclosure is directed to linear-array oxygen sensors for measuring oxygen partial pressure (pO₂). In some examples, the oxygen sensors may be formed or disposed along a long axis of a needle to obtain pO₂ readings at multiple points along the long axis of the sensor.

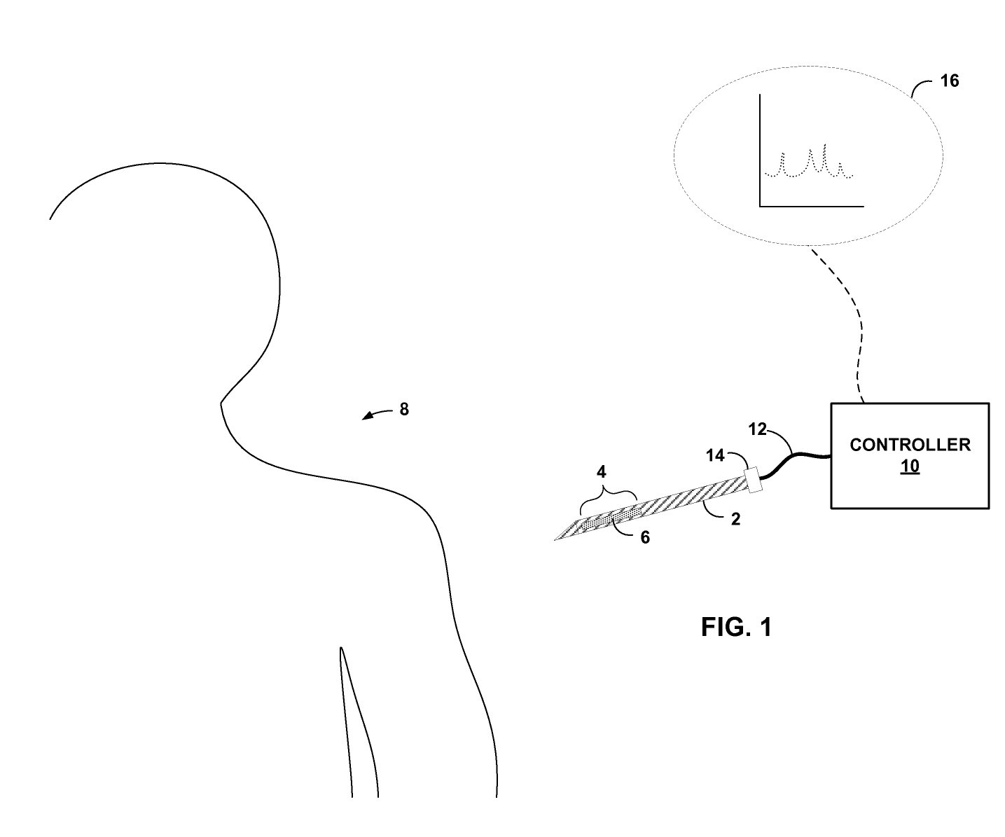

FIG. 1 is a conceptual diagram illustrating an example system including a needle-mounted oxygen sensor for measuring the pO₂ of tissue of a patient. In the example of FIG. 1, needle 2 includes a window 4 on a side of the needle in which an oxygen sensor 6 is disposed axially along a shaft (long axis) of the needle. The needle-mounted oxygen sensor 6 may be utilized in the disclosed techniques for measuring a depth profile of oxygen levels in the tissue or blood of a patient 8 at a series of positions along the shaft. An example implementation of oxygen sensor 6 may include, for example, one or more polymeric optical waveguides, where at least one of the waveguides includes an elongated channel of oxygen-sensing dye embedded in the polymer of an inner core. In example implementations, a top portion of the inner core is configured to face outwardly from window 4 of needle 2, and a polymeric outer core of the waveguide surrounds side and bottom portions of the inner core. In some examples, a polymer of the inner core may have an index of refraction that is higher than an index of refraction of the polymer of the outer core of the waveguide and higher than an index of refraction of the human tissue of patient 8. As described in further detail below, in some examples needle 2 includes multiple waveguides embedded therein for delivery of light signals to and/or from sensor 6. Further, in some examples, window 4 may have a length L of about 20 mm.

Once percutaneously inserted, oxygen molecules at different depths in the tissue of patient 8 are exposed to and diffuse through polymeric layers of the optical waveguide of oxygen sensor 6, and interact with the oxygen-sensitive dye embedded in the inner core of the optical waveguide. In some examples, a low coherence light source may be used to generate a time-varying and space-localized interference pattern to excite the oxygen-sensitive dye. Light from the low coherence light source (e.g., a continuous wave laser, not shown in FIG. 1) may be delivered to the optical waveguide of sensor 6 by one or more optical fibers 12. In some examples, one or more optical fibers 12 may be optically and mechanically coupled to the waveguide(s) of needle 2 via a coupler 14 attached to a proximal end of needle 2 or a distal end of one or more fibers 12 (see FIG. 1).

In some examples, a controller 10, as shown in FIG. 1, or components thereof, may operate to direct two waves of light from the low coherence light source at varying relative time delay so as to interfere at different sections of the optical waveguide of oxygen sensor 6. In some of these examples, a first wave may be delayed (e.g., its optical path length increased) before entering the waveguide, while the phase of a second wave may be modulated (e.g., shifted from zero radians to pi radians and back from pi radian to zero radians), to cause the waves to generate an interference pattern within the waveguide along a defined section of coherence between the waves. For example, by controlling the relative delay between the two light waves, controller 10 may cause an interference pattern to be generated at a particular point or a region of the waveguide of sensor 6 at which the two waves (delayed and non-delayed) have substantially the same optical path length. As such, controller 10 may vary the time delay between the two light waves in a controlled manner so as to locate the interference pattern at the set of positions along sensor 6, thereby effectively scanning sensor 6 and developing a depth profile of oxygen levels in the tissue or blood of a patient 8 at a series of positions along needle 2.

In the described examples, the generated interference pattern caused by the interfering waves may excite the dye and cause characteristic signals to be emitted by molecules of the oxygen-sensing dye. For example, the interfering waves may generate an interference pattern of transient dark-to-bright or bright-to-dark spots (or speckles) as the phase of the second wave is shifted. A rise rate of emission from the oxygen-sensing dye corresponding to dark-to-bright spots of the interference pattern may differ from a rate of decay or relaxation of emission from the dye at bright-to-dark spots. Further, to continuously or iteratively scan along different sections of the axis of oxygen sensor 6, a time period of delay of the first wave of low coherence light may be modified to move the interference zone accordingly.

As described in greater detail herein, the sensing mechanism of the oxygen-sensing dye may be based on triplet-state (and phosphorescence) quenching of the dye by oxygen molecules. In some examples, the emitted signals may traverse the optical waveguide, exit the waveguide via one or more optical fibers 12, be filtered by a dichroic mirror, and be received by a photodetector of controller 10. One or more components of controller 10 (e.g., a processor) may analyze characteristic features (e.g., triplet state life-time or frequency) of the signals received from the dye to calculate and display to a user a chart 16 including a depth profile of pO₂ values of the tissue of patient 8, as shown in FIG. 1. As one example, a processor of controller 10 may utilize the Stern-Vollmer calibration equation to determine pO₂ values along a long axis of oxygen sensor 6, as further described below.

In other examples described in further detail below, a waveguide of oxygen sensor 6 disposed on or within needle 2 may include a plurality of reflecting objects at multiple points along a long axis of oxygen sensor 6. In some of these examples, transient absorption of an oxygen-sensing dye embedded in sensor 6 may be measured using a low coherence interferometry (LCI) technique. LCI may allow for measurement of optical reflection from multiple points along the sensor. The reflection signals may be used to calculate the transient absorption at multiple sections along the waveguide of sensor 6, values which are then used to calculate the triplet state life-time at each segment, which can be converted to pO₂ values using the Stern-Volmer calibration equation.

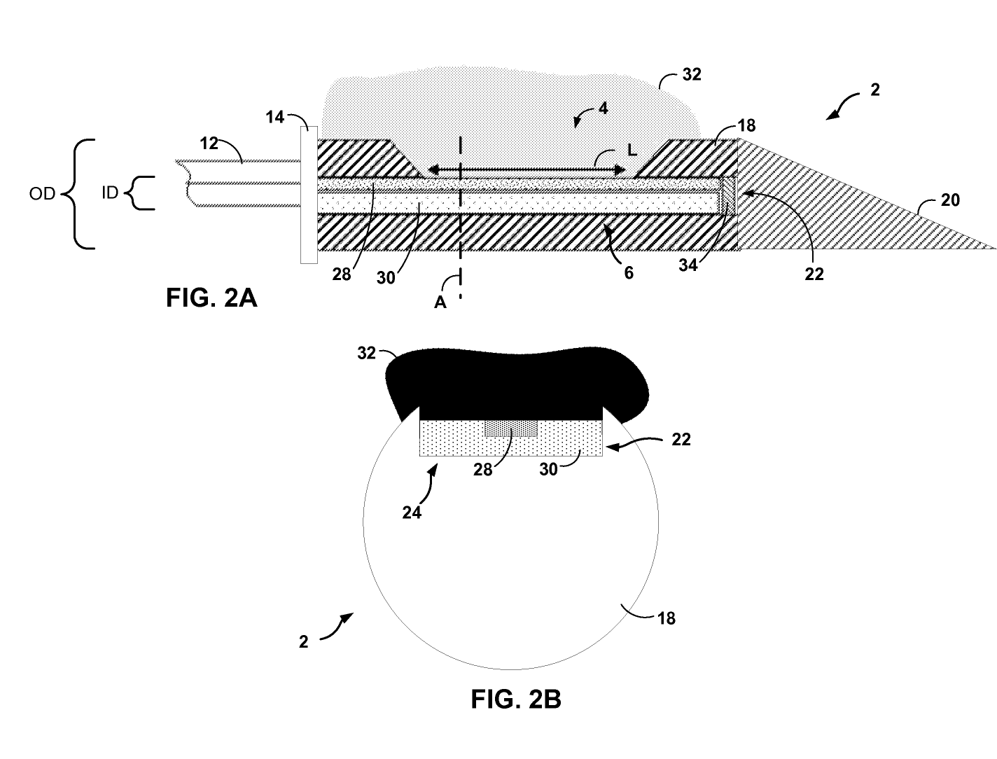

FIG. 2A is a conceptual diagram illustrating an axial cross-section of an example needle including an oxygen sensor. As shown in the example of FIG. 2A, needle 2 includes a shaft 18 and a bevel 20 defining at least a portion of a point of the needle. In some examples, one or more exterior surfaces of shaft 18 of needle 2 may define an outer diameter (OD) of shaft 18. The outer diameter of shaft 18 of needle 2 may vary, depending, for example, on the type of oxygen sensor formed on or housed within shaft 18 of needle 2. In some examples, an outer diameter of needle 2 may be less than about 0.5 millimeters (mm), such as about 0.3 mm. As described above, one or more exterior surfaces of shaft 18 of needle 2 may define window 4 in shaft 18, such that an interior core or trench 22 within needle 2 is exposed to the external environment via window 4 (see FIG. 2A).

Window 4 and trench 22 in needle 2 may be formed by any suitable technique, such as by machining. In some examples, bevel 20 of needle may be coreless. Further, shaft 18 and bevel 20 of needle 2 may be composed of any suitable biologically inert material, such as stainless steel, titanium, or the like.

As stated, one or more interior surfaces of shaft 18 may define interior core or trench 22 within shaft 18. Trench 22 may extend along a portion (or all) of a long axis of shaft 18 of needle 2. Trench 22 may be any suitable shape, such as cylindrical or an elongated rectangle, for receiving or otherwise containing a waveguide having an oxygen sensor 6. For example, trench 22 may have a height or inner diameter (ID) and be rectangular in axial cross section, as shown in FIG. 2A. In some examples, trench 22 has a substantially consistent (consistent or nearly consistent) dimension along its axial length. In other examples, dimensions of the cross-section of trench 22 may change along a long axis of needle 2. Trench 22 may take any suitable shape in radial cross section, such as a curved or polygonal shape, for example a circular or rectangular shape in radial cross section. FIG. 2B is conceptual diagram of a radial cross-section of the needle of FIG. 2A along line A. In the example needle 2 of FIG. 2B, trench 22 is shown as being rectangular in cross section. In general, trench 22 may be configured to house oxygen sensor 6. In some examples, as described below, multiple trenches may be formed in shaft 18 of needle 2 to house multiple optical waveguides of an oxygen sensor, such as oxygen sensor 6 or other configurations of oxygen sensors.

In some examples, as shown in FIGS. 2A and 2B, an oxygen sensor 6 may be formed on needle 2 or otherwise disposed along a long axis of needle 2, such that a long axis of oxygen sensor 6 and a long axis of needle 2 are substantially parallel (e.g., parallel or nearly parallel). In some examples, oxygen sensor 6 may include an optical waveguide 24 configured to deliver light and an optical coupler 26. In some of these implementations, optical waveguide 24 may include at least an inner core 28 and an outer core 30, each of which extends along a common long axis of waveguide 24 and needle 2. For example, outer core 30 may surround at least a portion of inner core 28 along the long axis of needle 2. For example, as shown in FIGS. 2A and 2B, outer core 30 may surround side portions and a bottom portion of inner core 28. In this way, a top portion of inner core 28 remains exposed to the external environment. For example, when needle 2 is percutaneously inserted, inner core 28 containing an oxygen-sensing dye may be in direct contact with tissue 32 via window 4 in shaft 18 of needle 2 (see FIGS. 2A and 2B).

In other examples, outer core 30 may fully encapsulate inner core 28 along some or all of an axial length of waveguide 24. In general, inner core 28 and outer core 30 may be configured to direct the delivery of light in certain ways, such as to direct light of particular wavelengths into the inner core 28 or outer core 30, where such light may be subject to total internal reflection within the respective core.

For needle-mounted implementations of the disclosed techniques, the dimensions of waveguide 24 may vary depending on the size of the needle. In some examples, without limitation, the lateral dimensions (width and height) of waveguide 24 may range from several microns to several hundreds of microns. Further, an example axial length of waveguide 24 may range from about 5 millimeters (mm) to about 50 mm.

In some examples, inner core 28 and outer core 30 of optical waveguide 24 may include one or more polymers. In general, suitable polymers for cores of optical waveguide 24 include polymers having high permeability and high oxygen diffusivity. In this way, for example, when needle 2 is percutaneously inserted into a human patient or other animal subject, oxygen molecules present in tissue 32 or blood of the patient may diffuse into inner core 28 of optical waveguide 24 of oxygen sensor 6 via window 4 in needle 2. Again, in other examples, depending on the configuration of the oxygen sensor, oxygen molecules may diffuse first into and through an outer core then diffuse into an inner core of the waveguide. Upon insertion, different volumes of oxygen molecules may diffuse into optical waveguide 24 along a length L of window 4 based on the amount of oxygen present in particular segments of tissue 32, as shown in FIG. 2A.

For example, silicone elastomers may be utilized to form polymer matrices of inner core 28 or outer core 30 that have a high oxygen permeability as part of a dyed, thin film polymer sensor. In some examples, silicone elastomers may have an oxygen diffusivity similar to that of tissue, such as D≈10⁻⁵ centimeters (cm)²/s. For example, a polydimethylsiloxane (PDMS)/platinum octaethylporphyrin (PtOEP) film of about 40 micrometers (μm) in thickness may have a response time of approximately 1 second (s), meaning that oxygen concentration in the sensor film will in that time reach a steady state having approximately the same oxygen concentration as that of dissolved oxygen in the tissue or blood of a patient.

In general, the response time of an oxygen sensor as disclosed herein (e.g., oxygen sensor 6 or 7) may be estimated by the equation: h²/D, where h is the thickness of the waveguide and/or oxygen-sensing portion of the waveguide and D is the diffusivity coefficient for oxygen in the polymeric material of the waveguide and/or oxygen-sensing portion of the waveguide. For example, when inner core 28 is disposed at a top portion of waveguide 24 such that a top surface of inner core 28 is exposed to tissue 32 of a patient, h may be a thickness of inner core 28 measured in a direction normal to a long axis of the oxygen sensor or needle 2. In one such example implementation, if a thickness of inner core 28 is at least about 10×10⁻⁶ meters (m) and a desired response time of the oxygen sensor is less than or equal to 10 seconds, a polymeric material of inner core 28 having a diffusivity coefficient of at least 10⁻¹¹ (m²/s) may be utilized according the disclosed techniques.

Moreover, in some examples, outer core 30 of waveguide 24 may include a first polymer having a lower refraction index than a second polymer of inner core 28. In this way, excitation light of particular wavelengths may be introduced into optical waveguide 24 and directed toward inner core 28 of waveguide 24. The first and second polymer may be different polymers, or the same or similar polymers with different additives affecting the index of refraction. Further, in the example of FIG. 2A, inner core 28 may include a second polymer having a refraction index higher than the refraction index of tissue 32, for example human tissue, so that tissue 32 may act as a guide for light reflecting within inner core 28. In this example, a first polymer of outer core 30 of waveguide 24 also may have a refraction index higher than human tissue 32. Likewise, in example implementations where an inner core is encapsulated within an outer core of the optical waveguide and the outer core is exposed to human tissue via a window in the needle, a first polymeric material of the outer core may have a refraction index higher than human tissue, while a second polymeric material of the inner core has a refraction index higher than the first polymeric material of the outer core.

In some examples, outer core 30 may include an outer layer that covers an inner core 28 and a bottom layer that underlies inner core 28. In some of these examples, the outer layer and bottom layer of outer core 30 may be composed of the same material. In other examples, the bottom layer may be composed of a third polymer that also has a lower refraction index than a polymer of inner core 28.

In some examples, elements of oxygen sensor 6, such as polymeric layers forming inner core 28 and outer core 30 of waveguide 24, may be formed by deposition on needle 2 using any number of suitable techniques. For example, a polymeric waveguide 24 may be constructed using additive manufacturing or photolithography. In some examples, oxygen sensor 6, or components thereof, may be formed and deposited directly along shaft 18 of needle 2. In other examples, oxygen sensor 6, or components thereof, may be formed or assembled separately then disposed within core or trench 22 in shaft 18 of needle 2 and secured by friction fit or by adhesion to one or more interior surfaces of shaft 18 with a suitable material.

In some examples, in addition to or alternatively to trench 22, an exterior surface of shaft 18 of needle 2 may include an approximately flat portion (e.g., flat or nearly flat). The flat portion of shaft 18 may be formed by any suitable technique, such as machining. In such an example, the flat portion of shaft 18 may be configured to receive and support oxygen sensor 6.

As described above, in some implementations, a polymer of inner core 28 may contain an oxygen-sensing dye. For example, an oxygen-sensing dye may be embedded within a polymer of inner core 28 and shaped in the form of an elongated channel. The described oxygen-sensing dye may include phosphorescent dyes characterized by long emission lifetimes, from hundreds of nanoseconds to milliseconds, and even longer for certain dye types. For any specific phosphorescent dye, upon being excited, the lifetime and intensity of light emitted by the excited dye molecules may be affected by oxygen concentration in the surrounding environment. This relationship is described by the Stern-Volmer equation, described in greater detail below. The presence of oxygen may accelerate the rate of decay of, or quench, dye molecules from an excited state to a ground state.

The presence of oxygen may be specified in the disclosed techniques due to spin-exchange interaction induced between oxygen and a phosphorescent dye. For example, molecular oxygen has a triplet ground state that may match the triplet ground state of excited sensor dye. Measuring phosphorescence intensity (or a triplet lifetime) of signals generated by the excited dye may enable highly sensitive and highly specific tissue oxygen pO₂ measurement. For instance, in some examples, suitable oxygen-sensing, phosphorescent dyes may include dyes that, upon being excited by sufficient energy (e.g., light at a particular wavelength or range of wavelengths), emit light and have a triplet lifetime in the range of 1 microseconds (μs) to 1000 μs.

When the described photosensitizer or oxygen-sensing dye is excited by an optical pulse or continuous wave, in regard to a dye of high quantum yield for inter-system crossing, a significant part of the dye molecules may be excited to a first triplet state. The dynamics of the relaxation of the excited dye molecules back to a ground state depends on oxygen concentration. In some examples, the decay or relaxation is exponential. Dye molecules excited to a first triplet state may have a triplet state lifetime, designated as t₁. The lifetime t₁ depends on oxygen partial pressure according to the Stern-Volmer equation (1), as follows:

$$\frac{{I}_{0}}{{I}_{1}} = \frac{{I}_{0}}{{I}_{1}} = 1 + {k}_{O 2} {t}_{0} {pO}_{2} \qquad ( 1 )$$

where I₀ and I₁ signify phosphorescence intensity at zero oxygen and at oxygen partial pressure of pO₂, respectively, t₀ is the triplet state lifetime at zero oxygen conditions, t₁ is the triplet state lifetime at pO₂ partial oxygen pressure, and kO₂ is the quenching rate coefficient. During the decay (or relaxation) process, the optical absorption of the dye changes because the optical absorption of the excited triplet state molecules is determined by the triplet-triplet absorption spectrum, which differs from the singlet-singlet absorption spectrum of ground-state molecules.

In some example implementations, a polymer of inner core 28 of oxygen sensor 6 may contain one or more of methylene blue (MB), a platinum (Pt) complex of a porphyrin, a palladium (Pd) complex of a porphyrin, or a ruthenium (Ru)-based compound. In some examples, the lifetime of PtOEP may be about 90 microseconds (μs) at zero oxygen conditions and about 4 μs in an air environment. Further, PtOEP can be efficiently excited by a light source having a wavelength of about 520 nm, whereupon the excited dye molecules of PtOEP may produce a phosphorescence emission peak having a wavelength of about 640 nm. For example, in regard to the disclosed techniques, inner core 28 of oxygen sensor 6 may be doped with one or more of a platinum-octaethylporphine ketone (Pt-OEPK) or a palladium-octaethylporphine ketone (Pd-OEPK). Other suitable dyes are described in the article, Grist et al., Optical Oxygen Sensors for Applications in Microfluidic Cell Culture, Sensors, 2010, 9286-9316, the contents of which are hereby incorporated by reference in their entirety. The Grist article describes, among other things, example PtOEP, PtOEPK, PdOEP, PdOEPK, and Ruthenium-based compound dyes, excitation and emission peaks of respective dyes, various properties of oxygen-sensitive dyes (e.g., the unquenched lifetime of respective dyes and quantum yield), and example encapsulation matrices for dyes.

Returning to FIG. 2A, oxygen sensor 6 also may include an optical coupler 34 disposed at a distal end of waveguide 24 to reflect or otherwise direct light emitted from the optical waveguide back into the optical waveguide. In some examples, for example oxygen sensors including a single waveguide, optical coupler 34 may be configured to reflect light back through the waveguide. Optical coupler 34 may include, for example, a mirror or prism. For some example needles, optical coupler 34 may be embedded within core or trench 22 in shaft 18 of needle 2, and not aligned with window 4 in shaft 18.

In some examples, one or more optical fibers 12 may be coupled to needle 2 by coupler 14 configured to receive one or more optical fibers 12 and secure one or more optical fibers 12 to needle 2, as shown in FIGS. 1 and 2A. Coupler 14 also may be configured such that coupler 14 places one or more optical fibers 12 in optical alignment with a proximal end of optical waveguide 24. For example, one or more optical fibers 12 may be optically aligned with outer core 30 or first core 28 to deliver light into outer core 30 or first core 28, respectively. In addition, in some examples, a segment of optical fiber may be permanently installed within needle 2, such that a long axis of the segment is oriented in a direction substantially parallel to the long axis of the needle. For example, a segment of optical fiber may be permanently installed at or near a proximal end of a core or trench 22 within needle 2, such that the permanently installed segment optically connects one or more optical fibers 12 to optical waveguide 24.

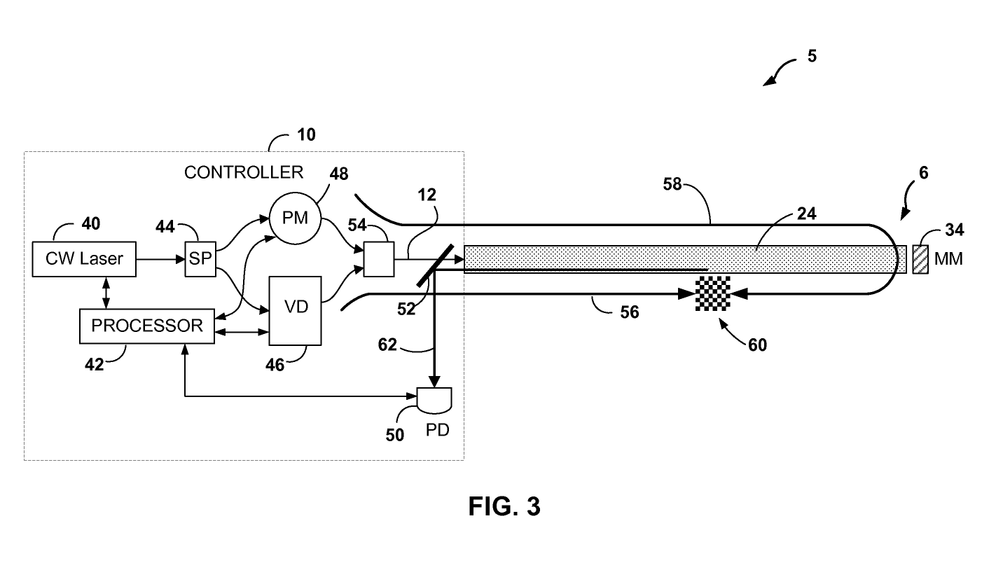

FIG. 3 is a schematic diagram illustrating an example system for measuring a depth-resolved pO₂ profile along sensor 6. Oxygen sensor 6 of an example system 5 may include the features described above, including, without limitation, optical waveguide 24 and optical coupler 34. As shown in FIG. 3 and described above with respect to FIG. 1, controller 10 includes various components designed to cause light waves to interfere and produce interference patterns along multiple sections of optical waveguide 24 of oxygen sensor 6. In some examples, controller 10 may include a housing that encloses various components necessary for controlling excitation light delivered to optical waveguide 24, receiving light emitted by dye molecules of the oxygen-sensing dye, and computing corresponding pO₂ values.

The various components of controller 10 of system 5 may be communicatively coupled as between one or more other components of at least controller 10, e.g., using optical, wired, or wireless communication, as appropriate. One or more optical fibers, wires, traces, or the like, may be disposed within controller 10 to connect the various components within controller 10, and to connect or couple components of controller 10 to external system components, such as oxygen sensor 6 or needle 2, as appropriate. In some examples, controller 10 may include a telemetry module and other components necessary for the secure wireless sending of signals and collected information to remote locations over a network, such as a remote server or computer. For example, a processor 42 and/or memory of controller 10 may be communicatively coupled to a telemetry module of controller 10 for wirelessly sending signals and/or information and receiving signals, such as instructions for operating various components of controller 10.

As shown in FIG. 3, in some examples, controller 10 may include a light source 40, such as a low or limited coherence light source. For example, light source 40 may include a continuous wave (CW) laser or free-space light delivery components and methods. In some examples, light source 40 may be optically coupled to an optical splitter (SP) 44 via an optical fiber, such as a single mode fiber. Controller 10 may further include an optical splitter (SP) 44 configured to receive excitation light from light source 40 and split the beam of excitation light in two beams or waves, each respective wave following a separate arm. For example, splitter 44 may utilize mirrors to split light from light source 40 into two waves having identical optical characteristics. In the described example, splitter 44 splits excitation light from light source 40 into a first wave directed toward a variable delay unit (VD) 46 along a first path and a second wave directed toward a phase modulator (PM) 48 along a second path.

In some examples, variable delay unit 46 and phase modulator 48 may be included within controller 10. Variable delay unit 46 may include any device configured to delay a wave of light by lengthening the optical path length of the wave. For example, variable delay unit 46 may include a series of translatable or rotatable mirrors mounted on a translation stage that can change the position of the translatable or rotatable mirrors with respect to a fixed set of mirrors and thereby change the length of the optical path of a wave inside variable delay unit 46. In such an example, a first wave received from splitter 44 may be delayed by a desired period of time before the first wave is introduced into waveguide 24.

Controller 10 also may include a processor 42 configured to control components of controller 10 and/or analyze signals received from the components or external sources. In general, processor 42 may control excitation light from light source 40 to cause an interference pattern at multiple sections along waveguide 24. A processor (e.g., processor 42), as described in this disclosure, may include one or more processors, such as one or more microprocessors, digital signal processors (DSPs), application specific integrated circuits (ASICs), field programmable gate arrays (FPGAs), programmable logic circuitry, or the like, either alone or in any suitable combination. Other components of controller 10 may be formed by suitable electrical and/or mechanical hardware elements, in combination with software or firmware, as appropriate. Some or all of the other components of controller 10 may be controlled by processor 42, or other suitable components of controller 10. For example, processor 42 may be configured to control (e.g., control settings or positions thereof) and communicatively coupled with light source 40, optical splitter 44, variable delay unit 46, phase modulator 48, combiner 54, dichroic mirror 52, detector 50 and/or any elements of the components, as appropriate. Portions of controller 10 may be implemented at least in part by a computing device (e.g., computer, laptop, mobile device, dedicated medical appliance) having one or more processors configured to execute software instructions.

In some examples, processor 42 may be configured to control variable delay unit 42 to delay a first wave of light by one or more selected periods of time before directing the delayed first wave to enter a proximal end of waveguide 24. As described in more detail below, in this example technique, adjusting the time of delay of the first wave corresponds to adjusting a location along waveguide 24 at which first wave may interfere with a second wave of light from light source 40. For example, delaying first wave for a shorter period of time may cause an interference pattern within waveguide 24 at a point or section of waveguide 24 that is closer to the distal end of waveguide 24, while delaying first wave for a longer period of time may cause an interference pattern at a point or region closer to the proximal end of waveguide 24.

Phase modulator 48 may include any device configured to shift the phase of a wave, such as a second wave received from splitter 44 via an optical connection thereto. For example, the phase modulator 48 may include an electro-optic crystal (such as lithium niobate LiNbO₃) that changes the phase difference of light between its input and output by applying an electrical field in the crystal. In addition or alternatively, as another example, phase modulator 48 may include an opto-mechanical medium (such as glass) in which a phase difference is introduced by applying mechanical stress. The mechanical stress can be generated by an external piezoelectric crystal.

In some examples, phase modulator 48 may be configured to shift the phase of a second wave of excitation light between two phases, such as from zero radians to pi radians, and back from pi radians to zero radians. Processor 42 also may be configured to control phase modulator 48 to shift or flip the phase of a second wave of excitation light from light source 40. For example, processor 42 may control phase modulator 48 to flip the phase of the second wave from zero to pi radians and from pi to zero radians.

In some examples, controller 10 may also include a combiner 54 configured to optically receive the first wave and second wave from the variable delay unit 46 and phase modulator 48, respectively, and recombine the two waves for optical delivery into waveguide 24. In some examples, combiner 54 may include components similar to or the same as splitter 44. For example, combiner 54 may be configured to deliver the recombined excitation light to waveguide 24 via one or more fibers 12, as described above. For example, as shown in FIG. 3, a proximal end of one or more fibers 12 may be coupled to combiner 54, while a distal end may be coupled to and optically aligned with waveguide 24. As another example, the recombined excitation light may be coupled to an output connector by a double-clad fiber coupler (DCC). Such a double-clad fiber may deliver the recombined excitation light to waveguide 24, optionally through a fiber connector (not shown in FIG. 3) at a box of controller 10.

Controller 10 also may include a detector 50, such as a photodectector (PD). Detector 50 may be configured to receive signals emitted by excited dye molecules of the oxygen-sensing dye that have exited waveguide 24. For example, detector 50 may receive both constant and oscillatory signals emitted from dye molecules that have been excited to a triplet state by excitation light traversing and interfering within waveguide 24 of oxygen sensor 6. In some examples, a dichroic mirror 52 may filter out extraneous wavelengths of light, such as excitation light from light source 40, and reflect or otherwise deliver selected wavelengths of emission light from dye molecules. In some examples, detector 50, processor 42, or one or more other suitable analog-to-digital conversion components of controller 10 may convert the analog emitted light signals, or characteristics thereof, to digital signals for further processing and computation of pO₂ values.

In operation, system 5 shown in FIG. 3 may resolve a spatial location of phosphorescence emission utilizing low coherence phosphorescence excitation (LCPE) delivered via one or more optional fibers 12.

In the example of FIG. 3, output of a limited coherence CW laser is split at splitter (SP) 44 into first and second waves having identical optical characteristics upon exiting splitter 44. Processor 42 then controls variable delay unit (VD) 46 to apply a variable delay to the optical path of the first wave, and two phase modulations are applied to the second wave by phase modulator (PM) 48 as the second wave continuously passes through PM 48 into combiner 54 then to waveguide 24. After being delayed for a selected period of time, the first wave exits variable delay unit 46 and also passes through combiner 54 then to waveguide 24 of oxygen sensor 6. The recombined excitation light of the first and second waves may be directed from combiner 54 to waveguide 24 one or more optical fibers 12, such as by a single double-clad fiber (DCF).

The optical path difference between the first wave and second wave, imparted by the delay of variable delay unit 46, may be set by controller 10 such that a delayed first wave 56 interferes with second wave 58 at a precise position along sensor 6 after second wave 58 is reflected from optical coupler 34 (e.g., a mirror (MM)). First wave 56 and second wave 58 of excitation light are shown schematically as arrows in FIG. 3. An interference pattern 60 is generated at a point or region of interference between first wave 56 and second wave 58 along waveguide 24. The location of the interference region along the waveguide sensor is determined by controller 10 by configuring the amount of time that light 26 is optically delayed.

Interference pattern 60 is generated at a point or region along the long axis of waveguide 24 where incoming delayed first wave 56 and reflected second wave 58 have substantially the same optical path length (the same or nearly the same). In other words, interference pattern 60 occurs where the two waves have propagated substantially the same distance since being split at splitter 44. In the example of FIG. 3, because VD 46 imparts a longer optical path length on first wave 56, it is not until second wave 58 is reflected back through waveguide 24 by mirror 34 and returns along waveguide 24 that the optical path lengths of the first and second waves match.

Interference pattern 60 occupies an interference zone or region in waveguide 24 In some examples, the interference region is between about 0.1 mm and 0.2 mm in size, depending at least in part on the type of light source 40 utilized (e.g., laser diodes) and the limit of its coherence length. Interference patterns 60 may include patterned light and dark spots (e.g., speckles) that switch locations at each phase flip of second wave 58 between zero and pi radians. The light and dark spots of interference pattern 60 may correspond to regions of constructive and destructive interference within interference pattern 60, respectively. Further, a stable speckle pattern may be generated when the first and second waves have substantially the same frequency (e.g., the same or nearly the same). For example, a selected region of about 0.2 mm along waveguide 24 (which may for example be exposed to tissue through a window of about 20 mm) may be analyzed by flipping the phase of second wave twice as described.

As described above, interference pattern 60 generated within a section of waveguide 24 may excite dye molecules of an oxygen-sensing dye embedded in a polymer of first core 28. Upon the flipping of second wave 58 between phases, and further due to the described intermolecular interactions between the dye molecules and oxygen molecules that have diffused into inner core 28, characteristic signals 62 (e.g., light) (see FIG. 3) are emitted by the dye molecules in response to the varying interference pattern 60. Although these phase modulations do not change the total intensity of the excitation light, they generate oscillatory phosphorescence emission because of the high non-linearity (saturation) between excitation of dye molecules of the oxygen-sensing dye and emission of light by the dye molecules. Transient emission from areas undergoing dark-to-bright change (e.g., a rise rate) has a different relaxation time constant than bright-to-dark spots (e.g., a relaxation rate) because the respective rates depend on illumination intensity. This difference results in a transient and characteristic emission of the total phosphorescence at each phase flip (e.g., light emitted at double the frequency of the phase modulation), which may be received and/or detected by detector 50 and analyzed by processor 42. The periodic phase modulations may generate double-frequency phosphorescence emission only in the interference zone.

As stated, light emitted from excited dye molecules quenched by oxygen in the interference region is different than light emission from other regions along waveguide 24 (and in some implementations, along needle 2) because the intensity of the light emission increases and then decreases (spikes) after each phase flip of second wave 58 by phase modulator 48. In some examples, because there are two phase flips in each period (zero to pi and then pi to zero), the detected intensity spikes twice in each period of the periodic phase modulation. This characteristic response (e.g., two spikes per period) can be tracked by either time-domain or frequency-domain analysis of the emission signal. In the latter, the emission signal may appear as a response in the double frequency and in higher even multiples of the basic modulation frequency. The dominant signal may be in the second harmonic, while the higher harmonics (4th, 6th, etc.) may appear weaker.

While the disclosed techniques may be applied to dyes having any lifetime, in some examples, oxygen-sensing dyes having a triplet lifetime of between 1 microsecond (μs) and 1,000 μs may enable ease of implementation of system components, such as the phase modulator and photodetector, which may have some limits in their response time. In some examples, selected oxygen-sensing dyes utilizing the disclosed techniques may have a lifetime ranging from between about 50 μs and about 300 μs.

Phosphorescence from regions outside the interference zone is not affected by the phase modulation because the first and second waves are not coherent with matching optical path lengths outside of the interference zone. Therefore, the signals emitted by dye molecules outside of the interference zone appears steady when received by detector 50. Further, the speckle contrast apparent in the interference region will diminish to zero as the path difference between the two waves increases. Such a speckle contrast may be negligibly small when the path difference is much larger than the coherence length of light source 40. In some examples, phosphorescence emission from all regions may be guided by waveguide 24 and collected by a multi-mode section of a double-clad fiber. For example, phosphorescence emission (steady and oscillating portions) may be collected by the multi-mode part of the DCF, separated by a dichroic mirror (DM) 52, and directed to a photodetector (PD) 50.

Thus, a complete scan of the active region (e.g., portions of oxygen sensor 6 having oxygen diffused therein) along the length of waveguide 24 is achieved by changing the optical path difference between the two waves traversing the respective two arms before the waves are recombined to enter waveguide 24. Multiple time periods of delay may be implemented by a computer-controlled stage (e.g., processor 42 of controller 10 controlling variable delay unit 46). Further, a scan of active regions of waveguide 24 may take place after complete stabilization of oxygen in waveguide 24 (several response times). In some examples, a dwell time in each scan position of a section of waveguide 24 may be about 0.1 second, although other time periods may be suitable. In general, and as discussed above, a response time of oxygen sensor 6 to changes in oxygen content or changes in environment (such as a change while inserting needle 2 into tissue) may be determined by the diffusion time of oxygen through the polymer waveguide. Thus, response time may be a function of both the dimensions of the waveguide and the type of polymer used. As one example, a PDMS polymer waveguide that is 40 microns in size may yield a response time of about 1 second.

In one example implementation, an oxygen sensitive PDMS/PtOEP film forms a thin and long optical waveguide. Light from a limited-coherence excitation source (such as Osram LP520) is split into two arms. One arm is optically delayed, and the other is phase modulated, before the two waves are combined again and coupled to the sensor waveguide. A mirror at a distal end of the waveguide opposite from the excitation source reflects light and causes the reflected light to interfere in the waveguide with incoming light.

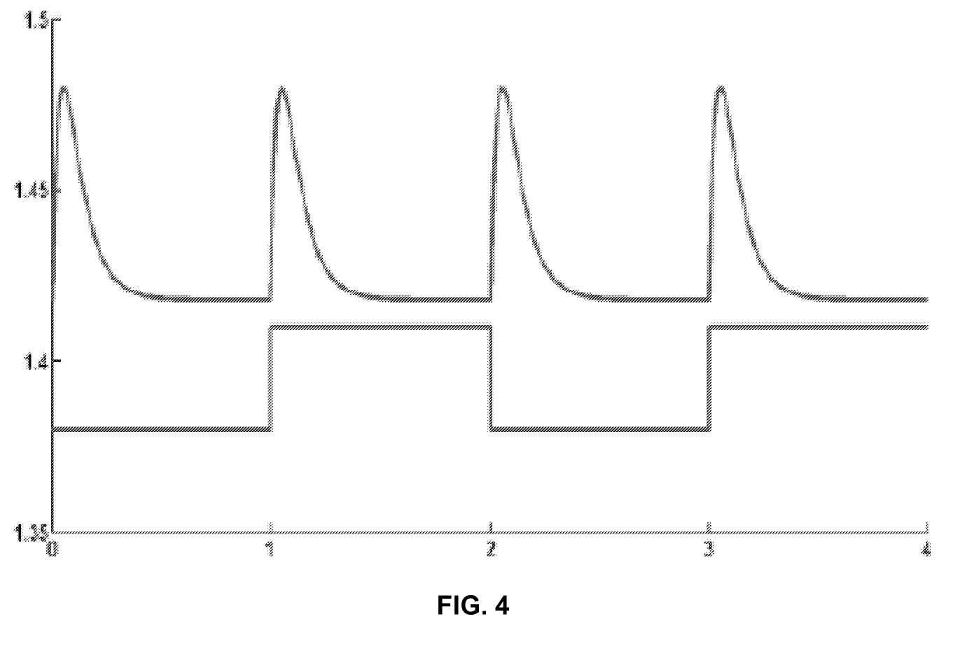

FIG. 4 is a chart illustrating a simulated result of interference zone emission produced by the system of FIG. 3. Phase modulation is shown as the bottom waveform, emission of excited dye molecules is shown as the top waveform. The simulation assumes that first and second waves enter a waveguide and are each reflected within the waveguide. The simulation also assumes that the incoming first and second waves have a same amplitude. As described above, interference is generated by a reflected second wave having a matching optical path length to an incoming delayed first wave. The upper waveform in FIG. 4 also shows the characteristic spike in the signal emitted by the dye molecules in response to a phase of the first wave being shifted between zero and pi radians.

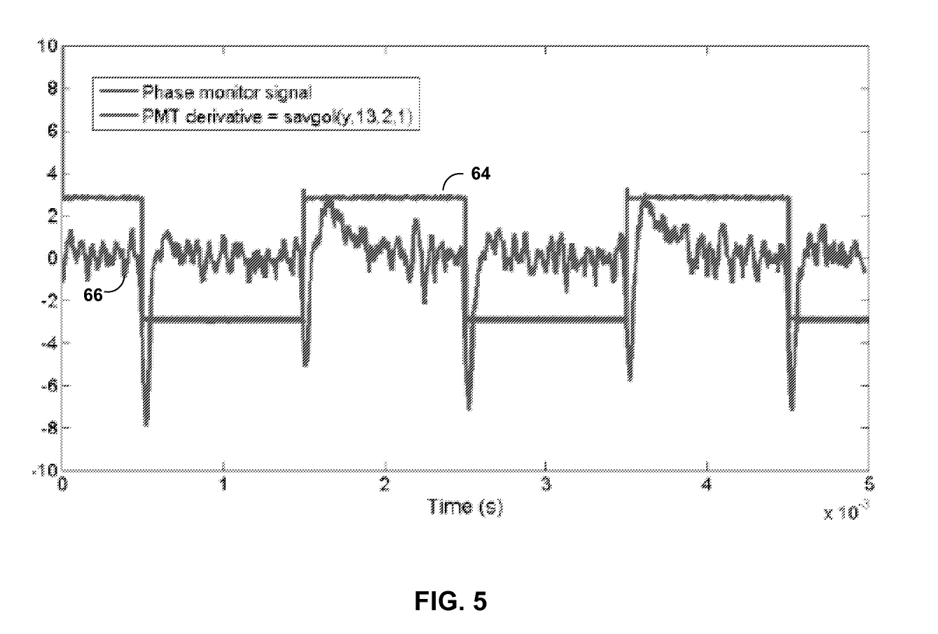

FIG. 5 is a chart illustrating experimental results showing a square-wave phase modulation and a phosphorescence signal double-frequency response. As shown in FIG. 5, the upper waveform 64 is a square-wave phase modulation, while the lower waveform 66 is the phosphorescence signal double-frequency response. The lower waveform 66 displays a characteristic spike in response to the phase modulation.

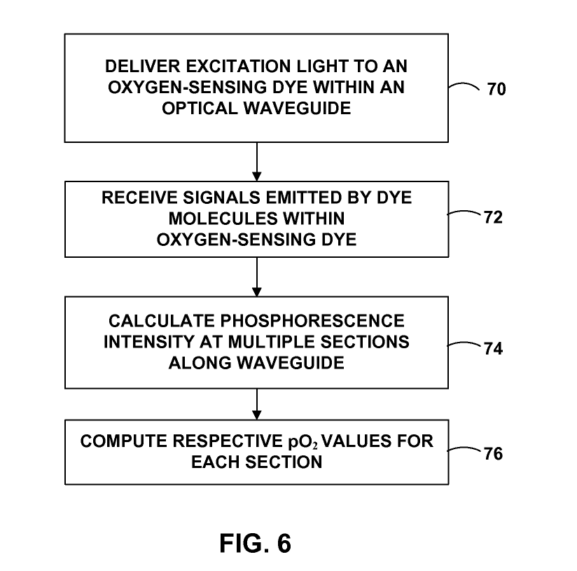

FIG. 6 is a flow diagram illustrating an example pO₂ measurement technique. As described above, for example with respect to operation of system 5 of FIG. 3, an example technique of this disclosure for measuring oxygen partial pressure (pO₂) at multiple points or sections along a needle may include delivering excitation light to oxygen-sensing dye 6 within optical waveguide 24 formed along shaft 18 of needle 2 (70). For example, delivering excitation may include delivering first wave 56 and second wave 58 to waveguide 24 as described above. The example technique may further include receiving, via at least optical waveguide 24, signals 62 emitted by dye molecules within the oxygen-sensing dye (72). For example, signals 62 emitted by the dye molecules may traverse toward a proximal end of waveguide 24, exit waveguide 24 via a multi-mode section of a double-clad fiber, be filtered by dichroic mirror 52, and received by detector 50, such as a photodetector.

The described example technique also may include calculating, based on the emitted signals 62, a phosphorescence intensity of emitted signals 62 at multiple sections along optical waveguide 24 (74). For example, detector 60 and/or processor 42 of controller 10 may perform such calculations. Further, the example technique may include computing respective pO₂ values for each section of waveguide 24 based at least on the respective phosphorescence intensity calculated at each section (76). For example, as described above, processor 42 may calculate a triplet state lifetime at each section of waveguide 24 and compute respective pO₂ values for each section based on the respective phosphorescence intensity and triplet state lifetime for each section using the Stern-Volmer calibration equation.

In some example techniques, an optical coupler (e.g., mirror) may be disposed at a distal end of waveguide 24, such that the example techniques further include, prior to delivering the excitation light to the oxygen-sensing dye: splitting the excitation light into first wave 56 and second wave 58; delaying first wave 56 by a first selected time period; and shifting the phase of second wave 58 (e.g., between zero and pi, then pi and zero radians), as described above. In some of these examples, delivering excitation light to the oxygen-sensing dye may include delivering the first and second waves of excitation light to the oxygen-sensing dye such that the phase-shifted second wave 58 continuously traverses waveguide 24 and is reflected back through waveguide 24 by mirror 34. In this way, reflected second wave 58 may interfere with delayed first wave 56 to cause interference pattern 60 within waveguide 24 that excites the dye molecules to emit signals 62 at one section of multiple sections that may be scanned along waveguide 24.

After causing a first interference pattern 60, an example technique also may include delaying first wave 58 by one or more additional selected time periods different from the first selected time period; again shifting the phase of second wave 58; and delivering first wave 56 delayed by the one or more additional selected time periods and the phase-shifted second wave 58 to the oxygen-sensing dye within optical waveguide 24. For example, to delay first wave 56 for a longer time period within variable delay unit 46, first wave 56 may be reflected between one or more additional mirrors or at one or more different angles between a series of mirrors within variable delay unit 46 before exiting the component. In such an example, processor 42 of controller 10 may be configured to control the number and positioning of mirrors, prisms, or the like within variable delay unit 46 to achieve a delay of a selected time period.

In some of these examples, the phase-shifted second wave 58 may continuously traverse waveguide 24 and be reflected back through waveguide by mirror 34, so that the reflected second wave 58 interferes with first wave 56 delayed by the one or more additional selected time periods to cause one or more additional interference patterns 60 that excite the dye molecules to emit signals 62 (see FIG. 3) at one or more additional sections of the multiple sections along waveguide 24. In this way, scanning multiple points or sections of waveguide 24 in a short time period may allow for the efficient computation and/or display to a physician or patient 8 of chart 16 including a depth-resolved pO₂ profile of tissue 32 of patient 8 along window 4 of needle 2 (see FIGS. 1 and 2A).

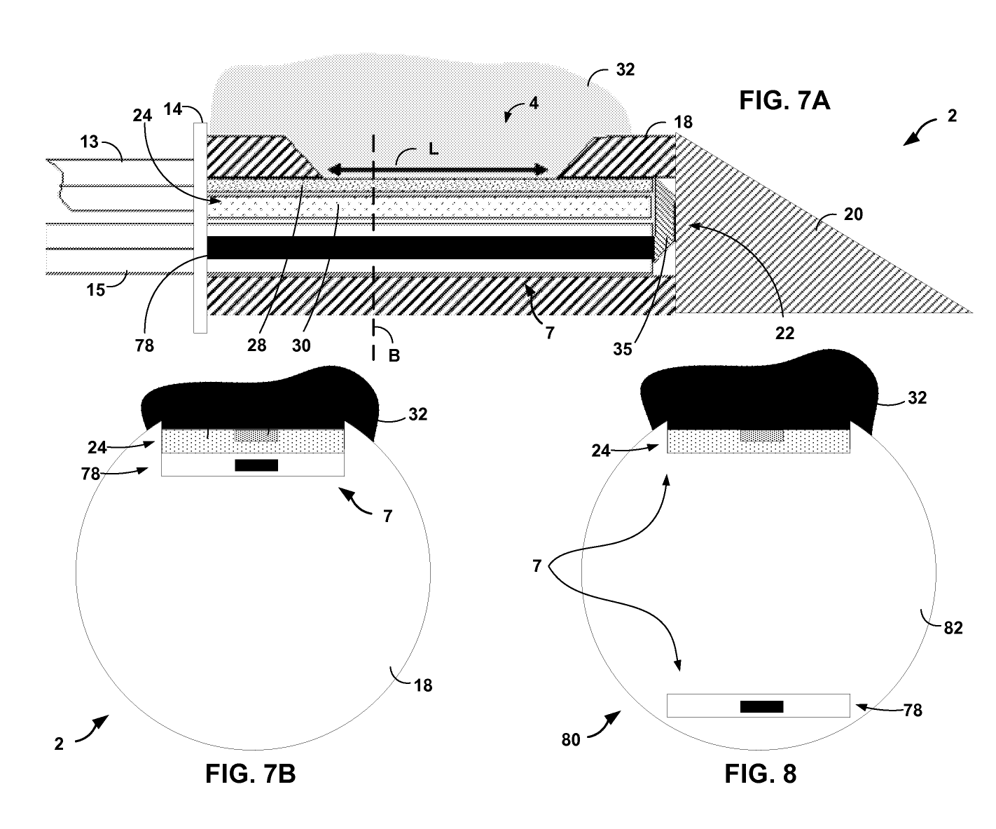

FIG. 7A is a conceptual diagram illustrating an axial cross-section of an example needle including an oxygen sensor with two optical waveguides. Needle 2, an oxygen sensor 7 disposed on needle 2, and other elements shown in FIG. 7A (and in other example needles below) may include features that are the same as or similar to those described above with respect to FIGS. 2A, 2B, and 3, unless otherwise noted. In the example needle of FIG. 7A, the core or trench 22 (or multiple cores or trenches) in shaft 18 of needle 2 may be configured to house oxygen sensor 7 having two optical waveguides in a variety of adjacent or spaced arrangements.

In general, a first optical waveguide of oxygen sensor 7 may include the features of optical waveguide 24 described above, such as outer core 30 and inner core 28 embedded with an oxygen-sensing dye. A second optical waveguide 78 of oxygen sensor 7 may include the same types of (or different) polymeric materials, an inner core, outer core, claddings, and geometric configurations as waveguide 24. However, in the example of FIG. 7A, second optical waveguide 78 does not include an oxygen-sensing dye embedded therein. In some examples, second optical waveguide 78 may be an optical fiber disposed in trench 22 of needle 2, either temporarily or permanently. In some implementations, the first optical waveguide and second optical waveguide of oxygen sensor 7 may be disposed along shaft 18 in respective orientations that are substantially parallel (e.g., parallel or nearly parallel).

In some example needles, the first optical waveguide may be disposed in a first trench defined within shaft 18, while second optical waveguide 78 may be disposed in a second trench defined within shaft 18. In some implementations, a portion of shaft 18 may extend in an axial direction along trench 22 and between the first optical waveguide and second optical waveguide 78, such that the first trench and second trench are disposed on either side of the portion of shaft 18 (not shown in FIG. 7A). The portion of shaft 18 separating the first and second trenches may vary in thickness depending on the placement of the first optical waveguide and second optical waveguide 78 along shaft 18 of needle 2.

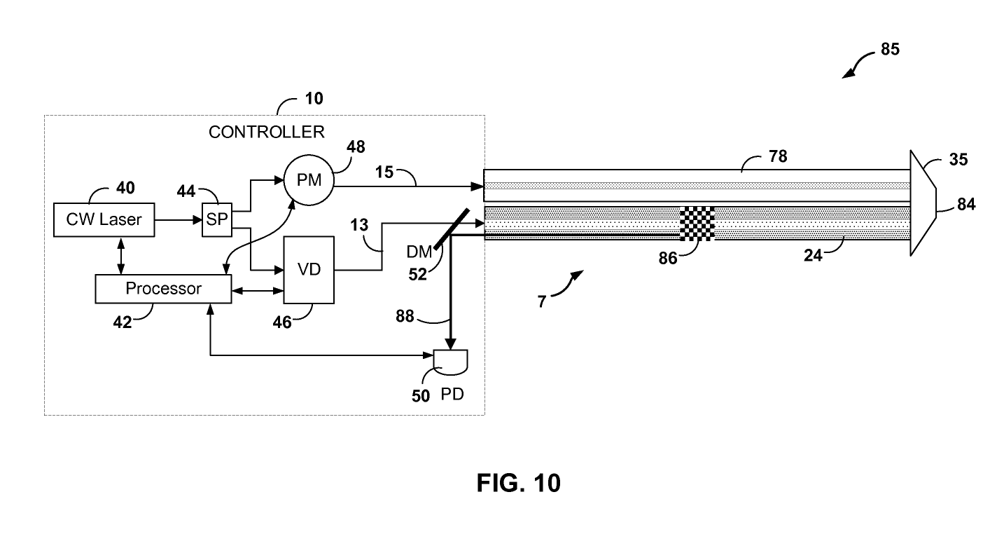

In the example shown in FIG. 7A, the first optical waveguide (e.g., optical waveguide 24) is positioned above second optical waveguide. An example system including an oxygen sensor with two waveguides (see FIG. 10) also may include two respective optical fibers, such that each optical fiber is coupled to a respective waveguide. As shown in FIG. 7A, a first optical fiber 13 may be optically coupled to a proximal end of the first optical waveguide, while a second optical fiber 15 may be optically coupled to second optical waveguide 78. The first and second optical fibers may optionally be mechanically coupled to needle 2 via coupler 14.

Oxygen sensor 7 also may include optical coupler 35 disposed at a distal end of the first optical waveguide and a distal end of second optical waveguide 78. Optical coupler 35 may include the same or similar features as optical coupler 34 described above. For example, optical coupler 34 may include one or more mirrors or prisms that are configured to reflect (or otherwise direct) light at least from second optical waveguide 78 into the first optical waveguide, so that two waves having substantially the same optical path length may interfere within the first optical waveguide to induce low coherence phosphorescence excitation, as described above. Utilizing two fibers to separately delivery the respective first and second waves of light into respective waveguides of an oxygen sensor, in connection with the pO₂ measurement techniques disclosed herein, may allow for an enhanced signal to background noise ratio within the first optical waveguide, as compared to examples including a single waveguide. For example, in the example needle of FIG. 7A, since only two waves traverse the first optical waveguide in opposite directions (e.g., a first wave entering the first waveguide at a proximal end from first optical fiber 13 and a second wave entering the first waveguide at a distal end after having been reflected), instead of four waves that may traverse optical waveguide 24 described in FIG. 2A, the interference pattern generated within the first optical waveguide may have a heightened contrast between dark and bright regions.

FIG. 7B is a conceptual diagram illustrating a radial cross-section of the needle of FIG. 7A along line B. As stated, the first and second optical waveguides of oxygen sensor 7 disposed or formed along needle 2 may be placed in a number of different adjacent and spaced arrangements within one or more trenches in shaft 18 of needle 2. In some examples, the first optical waveguide (e.g., waveguide 24) and second optical waveguide 78 may be disposed in a stacked arrangement, such that a bottom portion of one of the first and second optical waveguides is disposed above a top portion of the other of the first and second optical waveguides. For example, as shown in FIG. 7B, the first optical waveguide may be disposed adjacent to and above second optical waveguide 78, such that inner core 28 containing the oxygen-sensing dye is in direct contact with tissue 32 when needle 2 is percutaneously inserted.

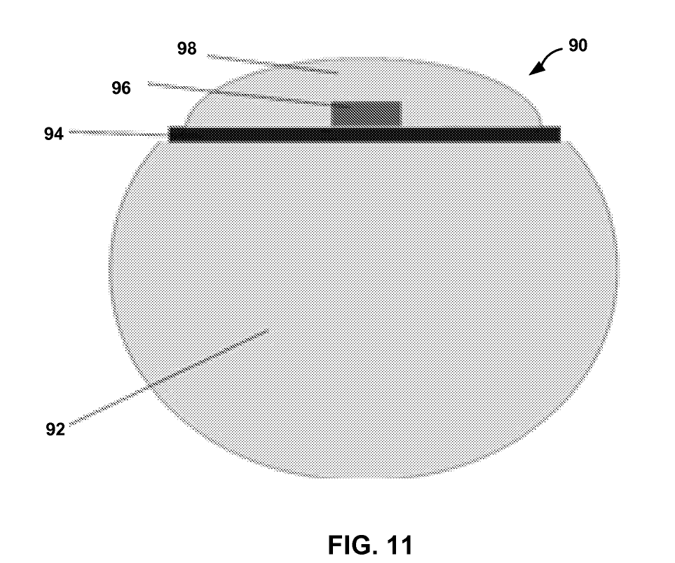

FIG. 8 is a conceptual diagram illustrating a radial cross-section of another example needle including an oxygen sensor with two waveguides. For example, a shaft 82 of a needle 80 may include two trenches that extend along a long axis of needle 80 but are spaced apart from one another, such as at opposing sides of needle 80 as shown in FIG. 8. A first trench may be formed in (e.g., machined) shaft 82 to receive a first optical waveguides (e.g., waveguide 24), and a second trench may be formed to receive or house (e.g., encapsulate exterior surfaces of) second optical waveguide 78. As shown in FIG. 8, a window 81 formed in shaft 82 of needle 80 may allow an inner core containing an oxygen-sensing dye to come in direct contact with tissue 32, such as human tissue. Window 81 may be formed in a same or similar manner, and include the same or similar features, as window 4 of needle 2 described above.

Thus, in the example of FIG. 8, oxygen sensor 7 includes the first optical waveguide and second optical waveguide disposed in a stacked arrangement, with a thick portion of shaft 82 extending therebetween along a long axis of needle 80. Further, an optical coupler (not shown) may extend between the spaced apart first and second waveguides and be configured to direct light at least from second waveguide 78 to the first optical waveguide (e.g., waveguide 24).

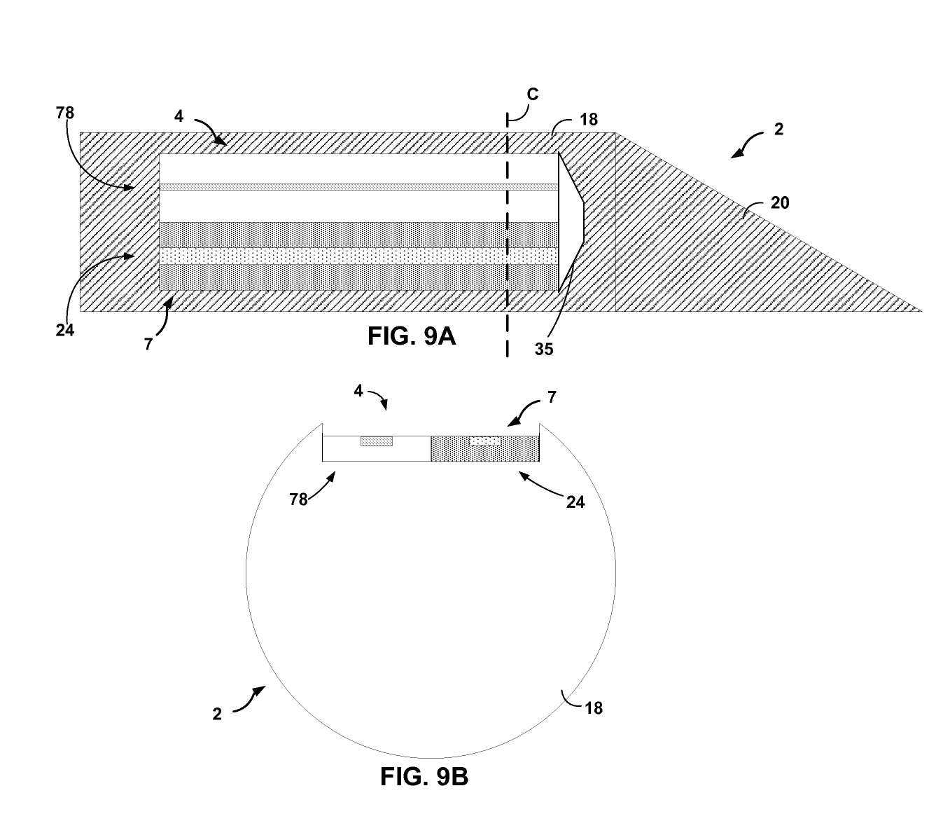

FIG. 9A is a top view of a conceptual diagram of an axial cross-section of another example needle including an oxygen sensor with two waveguides in a side-by-side arrangement. As shown in FIG. 9A, another example arrangement of first and second waveguides of oxygen sensor 7 disposed along needle 2 may include a side-by-side arrangement, such that a side portion of one of the first and second optical waveguides is disposed adjacent to a side portion of the other of the first and second optical waveguides. For example, as shown in FIG. 9A, a first optical waveguide, such as optical waveguide 24 previously described, may be positioned to the right of second optical waveguide 78 from a top view through window 4 in needle 2. In other implementations, a first waveguide resembling waveguide 24 may be positioned to the left of second waveguide 78 in a side-by-side arrangement. First and second waveguides may be disposed in a single trench or in respective separate trenches, as described herein. Similar to other examples, optical coupler 35 may be disposed at a distal end of the substantially parallel first and second waveguides, to guide light from second optical waveguide 78 to the first optical waveguide of oxygen sensor 7.

FIG. 9B is a radial cross-section of the example needle of FIG. 9A along line C. As shown in FIG. 9B, first and second optical waveguides of an oxygen sensor, such as waveguide 24 and second optical waveguide 78 of oxygen sensor 7, respectively, may be disposed in a side-by-side arrangement. The two waveguides may be disposed or embedded in a single trench formed along a portion of shaft 18, or alternatively, in respective trenches separated by a portion of shaft 18 that extends between the trenches.

FIG. 10 is a schematic diagram illustrating another example system for measuring a depth-resolved pO₂ profile. In general, the components and features of an example system 85 of FIG. 10 may be the same as or similar to those described above with respect to system 5 of FIG. 3, unless otherwise noted. For example, components of FIG. 10, such as processor 42, light source 40, optical splitter 44, variable delay unit 46, phase modulator 48, dichroic mirror 52, and photodetector 50 may be housed within a box of controller 10. Moreover, processor 42, alone in or combination with other components of controller 10 or external components, may be configured to control some or all of the other components of controller 10 of system 85. However, system 85, as shown in FIG. 10, does not include combiner 54 of controller 10 of system 5.

As described above, system 85 including oxygen sensor 7 with two waveguides also can include separate first and second optical fibers, each optically coupled to a respective waveguide. For example, as shown in FIG. 10, first optical fiber 13 may optically couple variable delay unit 46 to the first optical waveguide (e.g., waveguide 24). Further, second optical fiber 15 may optically couple phase modulator 48 to second optical waveguide 78.

Oxygen sensor 7 of system 85 also includes optical coupler 35 (e.g., a prism, one or mirrors, or a 180° mirror), which is configured at least to reflect (or otherwise direct) phase-modulated light from second optical fiber 78 to waveguide 24, as indicated by arrow 84. For example, optical coupler 35 may redirect a phase-modulated second wave to enter a distal end of waveguide 24 such that the redirected second wave interferes with a delayed first wave within waveguide 24 to cause an interference pattern 86. Again, light and dark regions of interference pattern 86 may have a heightened contrast as compared to interference pattern 60 of system 5, for reasons described above.

Apart from the second wave entering waveguide 24 at a distal end of waveguide 24 upon exiting second optical waveguide 78, an example technique for determining a depth-resolved pO₂ profile via system 85 may be substantially the same as the technique described above with respect to system 5. For example, interference pattern 86 may excite dye molecules of an oxygen-sensitive dye embedded in a polymeric material or matrix within an inner core of the first optical waveguide of oxygen sensor 7. As described in greater detail above, in some of these examples, the dye molecules may be excited from a ground state to a triplet state and relax from the triplet state to the ground state at different rates. Further, when phase modulator 48 flips the phase of a second wave, characteristic features of signals 88 (e.g., light) emitted from the dye molecules may be tracked. For example, signals 88 may include spikes of intensity or double frequency in an interference region along a long axis of oxygen sensor 7 in response to interference pattern 86.

In another example pO₂ measurement technique of this disclosure, a needle-mounted low coherence interferometry reflectors array may be utilized. FIG. 11 is a conceptual diagram of a radial cross-sectional view of an example linear-array oxygen sensor including a double-cladding waveguide disposed on a needle. In this example, an oxygen sensor 90 is designed as a double-cladding optical waveguide mounted on a substantially flat portion of needle 92 (e.g., a stainless steel needle). A bottom layer 94 containing a polymer may be deposited on needle 92 in some examples. Further, an inner core 96 containing a polymer, formed on at least a portion of bottom layer 92, may be stained with an oxygen-sensing dye (a photosensitizer) and may include a plurality of reflecting objects distributed along the long axis of the waveguide, e.g., along the axis parallel to needle 92. These reflectors can be realized by short sections of modified (increased or decreased) cross-sectional area. In some examples, the waveguide may include an outer core 98 containing a polymer, where outer core 98 is designed for delivering excitation light to the dye in inner core 96. Outer core 98 also provides mechanical protection to inner core 96.

In other example implementations, inner core 96 may be positioned such that a top portion of inner core 96 is exposed to the external environment when needle 92 is percutaneously inserted into tissue, while outer core 98 may surround at least side portions of inner core 96. In some of these examples, outer core 98 may be continuous with and serve as bottom layer 94.

In one example, oxygen sensor 90, as shown in FIG. 11, may be fabricated along a shaft of stainless steel needle 92, providing mechanical support and rigidity for tissue penetration. A flat portion of the exterior surface of needle 92 may be polished. In the described example, the polymer of bottom layer 94 may be a low refraction index polymer, such as Poly(methyl methacrylate) (PMMA). The polymer of inner core 96 may be a high refractive index polymer (such as polystyrene (PS)) that is doped by a photosensitizer dye (such as methylene blue (MB)). Outer core 98 may be designed for delivering excitation light to inner core 96. Further, outer core 98 may include a low refractive index polymer, such as PMMA or PDMS. The environment external to oxygen sensor 90 when percutaneously inserted (e.g., tissue or blood) typically has a lower refraction index than the polymer of outer core 98, and thus the external environment functions as an outer cladding to the outer core 98 to guide light in outer core 98.

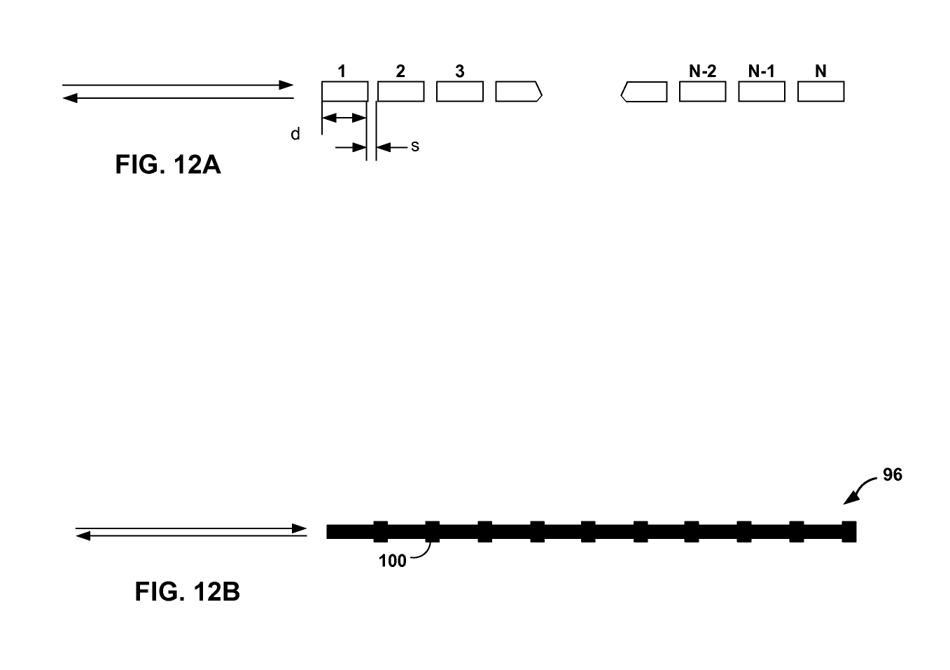

Example implementations of the waveguide core are illustrated in FIGS. 12A and 12B. That is, FIGS. 12A and 12B illustrate two example designs of inner core 96 of the linear array oxygen sensor 90 including a waveguide. The waveguide includes an array of reflectors 1, 2, 3 through N. As shown in FIG. 12A, these reflectors can be implemented as thin sections of low refraction index polymer separated by gaps of, for example, at least 0.2 mm. In the example of FIG. 12B, reflectors 100 may be implemented as sections of increased width in inner core 96. As one example, inner core 96 may be formed to have a thickness of approximately 0.040 mm, where the reflecting objects have a larger thickness of at least 0.042 mm.

As stated, inner core 96 may contain an oxygen-sensing dye embedded in a polymer matrix. The dye may have a relatively long triplet lifetime (for example, in the range of 50 μs to 300 μs) and a high quantum efficiency for inter-system crossing (for example, larger than 0.4). Suitable dyes include, for example: methylene blue (MB) and platinum (Pt) and palladium (Pd) complexes of porphyrins, such as platinum-octaethylporphine ketone (Pt-OEPK) and palladium-octaethylporphine ketone (Pd-OEPK).

A polymer matrix for inner core 96 may have a high oxygen diffusivity to enable a fast response time for diffusion of oxygen therethrough. In some examples, inner core 96 may have an oxygen diffusivity of at least 1.6×10⁻⁶ centimeters (cm)²/s. In some of these examples, a polymer having such an oxygen diffusivity coefficient may correspond to a response time (for sufficient diffusion of oxygen into the polymer) of less than 10 seconds using a waveguide of 40 μm in width. Polystyrene is one example of a polymer matrix material suitable as a waveguide material.

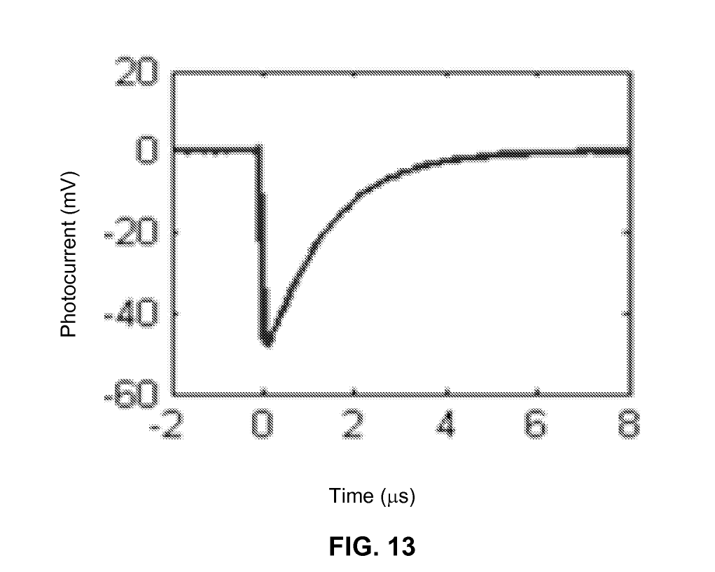

In some examples, bottom cladding layer 94 may include a thin polymer layer, such as Cytop or Poly(methyl methacrylate) (PMMA). Further, in some examples, inner core 96 may be covered by a top cladding layer (e.g., outer core 98) that includes a silicone elastomer, such as Polydimethylsiloxane (PDMS). Such silicone elastomers may have high oxygen permeability and are therefore suitable for fast response oxygen sensing. The described technique utilizing features described in FIGS. 11, 12A, and/or 12B may include a mechanism of oxygen transduction. For example, a photosensitizer dye may be excited by a short optical pulse. In the case of a dye of high quantum yield for inter-system crossing, a significant part of the dye molecules may be excited to the first triplet state. The dynamics of relaxation of the excited dye molecules back to the ground state depend on oxygen concentration surrounding the dye molecules. In one example, the decay is exponential having a lifetime t1. The lifetime t1 depends on oxygen partial pressure according to the Stern-Volmer equation, as described above. Again, as stated above, during the decay process the optical absorption of the dye changes because the optical absorption of the excited triplet state molecules may be determined by the triplet-triplet absorption spectrum, which differs from the singlet-singlet absorption spectrum of ground-state molecules. FIG. 13 is a graph illustrating an example measurement of transient optical absorption of a dye following excitation. In this example, the graph of FIG. 13 illustrates a photocurrent (in milli-volts, mV) of a photodiode measuring light transmission of a 810 nanometer (nm) CW diode laser through a sample of 40 molar (mM) methylene blue in a 10 mm square cuvette following pulse illumination by a 660 nm pulse.

A number density of molecules in an excited state can be described by the following expression.

nₑ(t)=n₀·exp(−t/t₁) (2)

Further, the transient absorption coefficient of the sample can therefore be expressed as:

μ(t)=n_{g}(t)σ_{g}+nₑ(t)σₑ=(n−nₑ(t))σ_{g}+nₑ(t)σₑ=nσ_{g}+nₑ(t)[σₑ−σ_{g}] (3)

where n, n_{g}, nₑ are the number density of total, ground state, and excited state molecules respectively, and σ_{g}, and σₑ are the absorption cross-sections of ground state and excited state molecules, respectively.

In certain example implementations, the waveguide may be designed as a double-cladding waveguide. Again, inner core 96 may contain a photosensitizer dye, while outer core 98 may deliver excitation light to inner core 96. In some examples, inner core 96 may include an array of reflectors, as described above. Each reflector may be, for example, designed as a narrowing or widening of the waveguide and the geometrical parameters of each reflector may be designed to generate an optical intensity reflection coefficient in the range of 0.001 to 0.1.

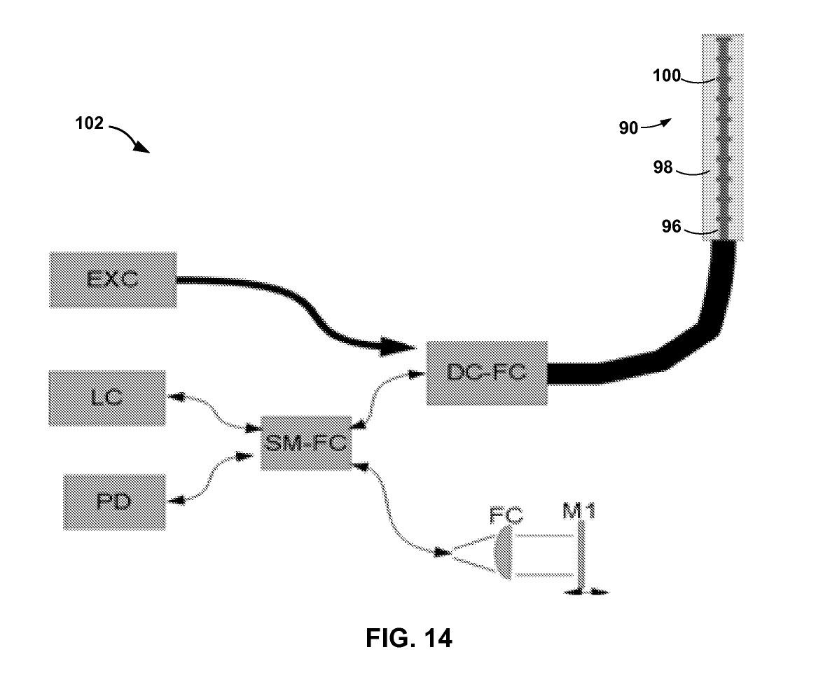

FIG. 14 is a schematic diagram illustrating an LCI-based system for measuring light transmission along the waveguide of an oxygen sensor at multiple points. For example, a system 102 may include a pulsed excitation light source (EXC), such as a laser diode driven by a current pulse or q-switched solid-state pulsed laser, may be optically coupled to outer core 98 of the waveguide-based oxygen sensor 90 through a double-clad fiber coupler (DC-FC). The pulse excitation induces transient absorption of the dye in inner core 96. An LCI configuration based on a fiber-optic Michelson interferometer may be used to measure the reflection from a single reflector of the reflectors 100 in inner core 96 of the waveguide.

In example system 102 shown in FIG. 14, a low coherence light source (LC) having a coherence length shorter than the distance between two consecutive reflectors (e.g., the distance designated by d+s in FIG. 12A) may be optically coupled to the core of the DC-FC coupler through a single-mode fiber coupler (SM-FC). The other arm of the SM-FC is connected to a scanning reflecting mirror (M1). The reflections from the sensor waveguide and from the mirror are coupled through the SM-FC to a photodetector (PD).

Further, in system 102, as shown in FIG. 14, a double-clad fiber oxygen sensor 90 may be connected to a double-clad fiber coupler (DC-FC). A pulsed excitation source (EXC) is coupled to outer core 96 of the sensor waveguide through the DC-FC. A low coherence light source (LC) is coupled to inner core 96 of the waveguide sensor through the DC-FC and a single mode fiber coupler (SM-FC). Further, a scanning mirror is connected to the other arm of the SM-FC. Reflections are routed by the SM-FC to a photodetector (PD).

The signal received at the photodetector in system 102 can be modeled as follows. The amplitude of the signal at the photodetector is a superposition of reflection from oxygen sensor 90 and the mirror M1. The reflection from the sensor waveguide is composed of separate reflection from each reflector. Denoting the reflection from the j-th reflector as rⱼ, the light reflection amplitude from the j-th reflector can be calculated by cascading the transmission coefficient of all reflectors in the path (having indices smaller then j), the reflection coefficient of the j-th reflector, and the attenuation due to dye along all sections in the path, as follows:

$${A}^{( m )} = d {A}_{i} ( t - {τ}_{m} ) {e}^{- i ( ω t - {k}_{0} 2 {z}_{m} )} + e {\sum }_{j = 1}^{N} {A}_{i} ( t - {τ}_{j} ) {a}_{j} {b}_{j} ( \tilde{t} ) {e}^{- i ( ω t - {k}_{0} 2 {z}_{j} )} \qquad ( 4 )$$

where A^{(m)} is the amplitude of the signal at the photodetector surface corresponding to mirror alignment at an equivalent optical path as that of reflector of index m, where τⱼ,zⱼ represent the optical time delay and path length corresponding to a signal reflected from reflector of index j, ω₀,k₀ are the center optical angular frequency and wave-number, N is the number of reflectors, d is the coupling coefficient of reflection from the mirror M1, and e is the coupling coefficient of light reflected from the sensor waveguide. Thus, aⱼ is the combined transmission coefficient of an optical signal reflected from the j-th reflector and is calculated in terms of the reflection coefficients rⱼ follows:

$${a}_{j} = {\prod }_{l = 1}^{j - 1} {( 1 - {r}_{l} )}^{2} {r}_{j} \qquad ( 5 )$$

The dye attenuation along the path reflected from the j-th reflector is bⱼ(\tilde{t}), which is a function of the “slow” time \tilde{t}. The term “slow” time here, refers to the time lapse since the pulse excitation and represents the slow time scale (microseconds) of variation of dye transient absorption due to triplet relaxation process:

$${b}_{j} ( \tilde{t} ) = {\prod }_{l = 1}^{j} {e}^{- {α}_{l} ( \tilde{t} ) 2 {s}_{l}} = {\prod }_{l = 1}^{j} {c}_{j} \qquad ( 6 )$$

where αⱼ(\tilde{t}),sⱼ are the transient absorption coefficient, and the length of the j-th section. It can be seen that

cⱼ=bⱼ/bⱼ₋₁ (7).

The intensity measured by the photodetector in system 102 is given by a time-average of the instantaneous intensity. The time-average is denoted here by:

$${〈 f 〉}_{\tilde{t}} = \frac{1}{T} ( f ) dt .$$

The intensity of the low coherence light source is denoted as I₀.

The photodetector intensity is:

$$〈 {\vert {A}^{( m )} \vert}^{2} 〉 = {dI}_{0} + {\sum }_{j = 1}^{N} {ea}_{j}^{2} {b}_{j}^{2} {I}_{0} + \frac{1}{2} {ea}_{m} {b}_{m} {I}_{0} \qquad ( 8 )$$

The first term is intensity of reflection from the mirror, the second term is a sum of intensity reflection from all reflectors 100, and the third term is the interference term of a single reflector (index m) and the mirror signal. The second term can be measured independently from the third term by aligning the mirror position such that none of the reflectors 100 have a matching optical path to the mirror reflection path (denoted as position “0”) and therefore the third term vanishes in this case.

The following steps illustrate an example measurement technique as described with respect to components of system 102. The technique may be implemented at least in part by a computing device (e.g., computer, laptop, mobile device, dedicated medical appliance) having one or more processors configured to execute software instructions. For example, the technique may be implemented using one or more features of controller 10 or processor 24, as described above.
1. Tune the mirror M1 to position “0”.
2. Measure and record the reference transient intensity waveform (i.e. the photodetector signal output as a function of the slow time \tilde{t}, the time after excitation pulse).
3. Tune the mirror M1 to position “m” (the position for which the optical time delay of the mirror signal matches that of the m-th reflector's signal) and record the waveform.
4. Subtract the reference waveform from the transient intensity waveform to obtain the difference waveform of reflector “m”.
5. Repeat step 4 for positions 1 through N.
6. Obtain the functions cₘ(\tilde{t}), for m in the range of 1 to N−1 by using equation 7 above.
7. Perform exponential curve fitting to the functions cₘ (\tilde{t}) and extract the decay time. Use the Stern-Volmer equation and calibration coefficients t₀, k₀ to determine the pO₂ value at segment positions 1 to N−1.

### Example 1

An oxygen sensor comprising: an optical waveguide comprising: a bottom polymer; an inner core polymer formed on at least a portion of the bottom polymer, wherein the inner core polymer contains an oxygen sensing dye and comprises a plurality of reflecting objects distributed along a long axis of the waveguide; and an outer core polymer covering the inner core and arranged to deliver optical excitation to the dye.

### Example 2

The oxygen sensor of example 1, wherein the oxygen sensing dye is embedded within the inner core and shaped in the form of an elongated channel extending along a long axis of the waveguide.

### Example 3

The oxygen sensor of example 1, wherein the channel is covered by the outer core polymer forming the optical waveguide as a double-cladding optical waveguide structure.

### Example 4

The oxygen sensor of example 1, wherein the bottom polymer and the outer core each have a refraction index lower than the inner core;

### Example 5

The oxygen sensor of example 1, wherein the outer core has a refraction index higher than human tissue.

### Example 6

The oxygen sensor of example 1, wherein the reflecting objects comprise thin sections of the inner core polymer.

### Example 7

The oxygen sensor of example 1, wherein the reflecting objects comprises thick sections of the inner core polymer.

### Example 8

The oxygen sensor of example 1, wherein the sensing dye has a triplet lifetime in the range of 50 μs to 300 μs.

### Example 9

The oxygen sensor of example 1, wherein the sensing dye comprises one or more of Methylene Blue (MB) or Platinum and palladium complexes of porphyrins including platinum-octaethylporphine ketone (Pt-OEPK) and palladium-octaethylporphine ketone (Pd-OEPK).

### Example 10

A needle comprising: a shaft; and the oxygen sensor of any of examples 1-9, wherein the bottom polymer is formed along the shaft of the needle such that the outer core is facing outward from the shaft.

### Example 11

The needle of example 10, wherein the shaft comprises an approximately flat portion on a side of the needle and extending along a long axis of the shaft, and wherein the bottom polymer of the oxygen sensor is formed along the flat portion of the needle.

### Example 12

A needle comprising a micro-waveguide structure mounted thereto and arranged to provide a profile of oxygen partial pressure (pO₂) along a shaft of the needle.

### Example 13

A method for measuring oxygen partial pressure (pO₂) at multiple points along a needle comprising: delivering, via the an optical wave guide formed along the needle, excitation light to an oxygen-sensing dye within the optical wave guide; receiving signals reflected from reflective objects within the optical wave guide; calculating, based on the reflected signals; the transient absorption at multiple sections along the optical waveguide; calculating a triplet state life-time at each section; and computing respective pO2 values for each section from the respective triplet state life-time.

### Example 14

The method of example 13, wherein computing respective pO2 values comprises: calculating a triplet state life-time at each section; and computing the respective pO2 values for each section based on the respective triplet state life-time computed for each of the sections.

### Example 15

The method of example 13, wherein computing respective pO2 values comprises computing the pO2 values by a Stern-Volmer calibration equation.

### Example 16

An oxygen sensor comprising: an optical waveguide comprising: an outer core comprising a first polymer; and an inner core comprising a second polymer, wherein the second polymer contains an oxygen-sensing dye; and one or more mirrors at a distal end of the waveguide to reflect light back through the waveguide. Example

### Example 17

The oxygen sensor of example 16, wherein the outer core and the inner core extend along a long axis of the waveguide, the outer core surrounds at least a portion of the inner core along the long axis, the oxygen-sensing dye is embedded within the second polymer and shaped in the form of an elongated channel, the first polymer has a refraction index that is lower than a refraction index of the second polymer, and each of the first and second polymers has a refraction index that is higher than a refraction index of human tissue.

### Example 18

The oxygen sensor of example 16, wherein the oxygen-sensing dye comprises one or more of: methylene blue (MB), a platinum (Pt) complex of a porphyrin, a palladium (Pd) complex of a porphyrin, or a ruthenium (Ru)-based compound.

### Example 19

The oxygen sensor of example 16, wherein the oxygen sensor further comprises a second optical waveguide, wherein the optical waveguide comprises a first optical waveguide, wherein the second optical waveguide is disposed along the shaft in an orientation substantially parallel to the first optical waveguide, wherein the one or more mirrors are disposed at a distal end of the first optical waveguide and a distal end of the second optical waveguide, and wherein the one or more mirrors are configured to reflect light at least from the second optical waveguide to the first optical waveguide.

The techniques described herein may be implemented in hardware, software, firmware, or any combination thereof. Various features described as modules, units or components may be implemented together in an integrated logic device or separately as discrete but interoperable logic devices or other hardware devices. In some cases, various features of electronic circuitry may be implemented as one or more integrated circuit devices, such as an integrated circuit chip or chipset.

If implemented in hardware, this disclosure may be directed to an apparatus such a processor or an integrated circuit device, such as an integrated circuit chip or chipset. Alternatively or additionally, if implemented in software or firmware, the techniques may be realized at least in part by a computer readable data storage medium comprising instructions that, when executed, cause one or more processors to perform one or more of the methods described above. For example, the computer-readable data storage medium may store such instructions for execution by a processor. Any combination of one or more computer-readable medium(s) may be utilized.

A computer-readable medium may form part of a computer program product, which may include packaging materials. A computer-readable medium may comprise a computer data storage medium such as random access memory (RAM), read-only memory (ROM), non-volatile random access memory (NVRAM), electrically erasable programmable read-only memory (EEPROM), flash memory, magnetic or optical data storage media, and the like. In general, a computer-readable storage medium may be any tangible medium that can contain or store a program for use by or in connection with an instruction execution system, apparatus, or device. Additional examples of computer readable medium include computer-readable storage devices, computer-readable memory, and tangible computer-readable medium. In some examples, an article of manufacture may comprise one or more computer-readable storage media.

In some examples, the computer-readable storage media may comprise non-transitory media. The term “non-transitory” may indicate that the storage medium is not embodied in a carrier wave or a propagated signal. In certain examples, a non-transitory storage medium may store data that can, over time, change (e.g., in RAM or cache).

The code or instructions may be software and/or firmware executed by processing circuitry including one or more processors, such as one or more digital signal processors (DSPs), general purpose microprocessors, application-specific integrated circuits (ASICs), field-programmable gate arrays (FPGAs), or other equivalent integrated or discrete logic circuitry. Accordingly, the term “processor,” as used herein may refer to any of the foregoing structure or any other processing circuitry suitable for implementation of the techniques described herein. In addition, in some aspects, functionality described in this disclosure may be provided within software modules or hardware modules.

Although specific embodiments have been illustrated and described herein, it will be appreciated by those of ordinary skill in the art that any arrangement that achieves the same purpose, structure, or function may be substituted for the specific embodiments shown. This application is intended to cover any adaptations or variations of the embodiments of the invention described herein. It is intended that this invention be limited only by the claims, and the full scope of equivalents thereof.

1. A needle comprising:
a shaft; and
an oxygen sensor comprising:
an optical waveguide formed along the shaft, wherein the optical waveguide includes an outer core comprising a first polymer and an inner core comprising a second polymer containing an oxygen-sensing dye; and
an optical coupler embedded within the shaft at a distal end of the waveguide to reflect light back through the waveguide.

2. The needle of claim 1, wherein the outer core and the inner core extend along a long axis of the waveguide, the outer core surrounds at least a portion of the inner core along the long axis, and the oxygen-sensing dye is embedded within the second polymer and shaped in the form of an elongated channel.

3. The needle of claim 1, wherein the first polymer has a refraction index that is lower than a refraction index of the second polymer, and wherein each of the first and second polymers has a refraction index that is higher than a refraction index of human tissue.

4. The needle of claim 1, wherein the oxygen-sensing dye has a triplet lifetime in the range of 1 microseconds (μs) to 1000 μs.

5. The needle of claim 1, wherein the oxygen-sensing dye comprises one or more of: methylene blue (MB), a platinum (Pt) complex of a porphyrin, a palladium (Pd) complex of a porphyrin, or a ruthenium (Ru)-based compound.

6. The needle of claim 1, wherein the shaft includes an approximately flat portion or a trench along a long axis of the shaft, and wherein the oxygen sensor is disposed at least partially along the flat portion or within the trench.

7. The needle of claim 1, wherein at least the inner core of the optical waveguide of the oxygen sensor faces outward from the shaft of the needle, and wherein the oxygen sensor is configured to provide a profile of oxygen partial pressure (pO₂) along a long axis of the sensor.

8. The needle of claim 1, wherein the oxygen sensor further comprises a second optical waveguide, wherein the optical waveguide comprises a first optical waveguide, wherein the second optical waveguide is disposed along the shaft in an orientation substantially parallel to the first optical waveguide, wherein the optical coupler is disposed at a distal end of the first optical waveguide and a distal end of the second optical waveguide, wherein the optical coupler comprises one or more mirrors, and wherein the one or more mirrors are configured to reflect light at least from the second optical waveguide to the first optical waveguide.

9. The needle of claim 8, wherein the first optical waveguide and the second optical waveguide are disposed in a stacked arrangement, such that a bottom portion of one of the first and second optical waveguides is disposed above a top portion of the other of the first and second optical waveguides.

10. The needle of claim 8, wherein the first optical waveguide and the second optical waveguide are disposed in a side-by-side arrangement, such that a side portion of one of the first and second optical waveguides is disposed adjacent to a side portion of the other of the first and second optical waveguides.

11. The needle of claim 8, wherein the first optical waveguide is disposed in a first trench defined within the shaft of the needle and the second optical waveguide is disposed in a second trench defined within the shaft, wherein a portion of the shaft extends in an axial direction between the first trench and the second trench.

12. A system comprising:
an oxygen sensor comprising:
an optical waveguide including an outer core and an inner core, the outer core comprising a first polymer and the inner core comprising a second polymer containing an oxygen-sensing dye; and
an optical coupler disposed at a distal end of the waveguide to reflect light back through the waveguide; and
a controller comprising a light source, a processor, and a detector,
wherein the light source is optically coupled to the waveguide of the oxygen sensor to deliver excitation light to the oxygen-sensing dye,
wherein the processor is configured to control a delivery time of the excitation light to the oxygen sensor to cause an interference pattern at multiple sections along the waveguide over a period of time, and
wherein the detector is configured to receive, via at least the waveguide, signals emitted by dye molecules of the oxygen-sensing dye at the multiple sections over the period of time in response to the interference pattern.

13. The system of claim 12, further comprising a needle, wherein the oxygen sensor is disposed along a shaft of the needle such that a long axis of the oxygen sensor is substantially parallel to a long axis of the needle.

14. The system of claim 12, wherein the controller further comprises:
a splitter configured to split the excitation light from the light source into a first wave and a second wave;
a variable delay unit configured to delay the first wave of excitation light;
a phase modulator configured to shift the phase of the second wave of excitation light; and
a dichroic mirror configured to direct the signals emitted by the dye molecules to the detector.

15. The system of claim 14, wherein the processor is further configured to control the variable delay to delay the first wave of excitation light for one or more selected periods of time and to control the phase modulator to shift a phase of the second wave of excitation light between zero and pi radians and between pi and zero radians, and wherein the optical coupler is configured to reflect the second wave back through the waveguide such that the reflected second wave interferes with the delayed first wave to cause the interference pattern.

16. The system of claim 14, the system further comprising one or more optical fibers, wherein the controller further comprises a combiner configured to recombine the delayed first wave and the phase-modulated second wave and deliver the recombined excitation light to the waveguide via the one or more optical fibers optically coupled to the combiner at a proximal end, and wherein a distal end of the one or more optical fibers are further configured to be mechanically coupled to one or more couplers and optically coupled to the waveguide of the oxygen sensor.

17. The system of claim 14, wherein the oxygen sensor further comprises a second optical waveguide, wherein the optical waveguide comprises a first optical waveguide,
wherein the variable delay unit is optically coupled to the first optical waveguide via a first optical fiber, wherein the phase modulator is optically coupled to the second optical waveguide via a second optical fiber, and wherein the optical coupler is configured to redirect the second wave to enter a distal end of the first optical waveguide such that the redirected second wave interferes with the first wave in the first optical waveguide to cause the interference pattern.

18. A method for measuring oxygen partial pressure (pO₂) at multiple points along a needle comprising:
delivering excitation light to an oxygen-sensing dye within an optical waveguide formed along a shaft of the needle;
receiving, via at least the optical waveguide, signals emitted by dye molecules within the oxygen-sensing dye;
calculating, based on the emitted signals, the phosphorescence intensity at multiple sections along the optical waveguide; and
computing respective pO₂ values for the sections based at least on the respective phosphorescence intensity at each of the sections.

19. The method of claim 18, wherein computing respective pO₂ values comprises:
calculating a triplet state life-time at each section; and
computing the respective pO₂ values, using a Stern-Volmer calibration equation, for the sections based further on the respective triplet state life-time calculated for each of the sections.

20. The method of claim 18, wherein a mirror is disposed at a distal end of the waveguide, the method further comprising, prior to delivering the excitation light to the oxygen-sensing dye:
splitting the excitation light into a first wave and a second wave;
delaying the first wave by a first selected time period; and
shifting the phase of the second wave,
wherein delivering excitation light to the oxygen-sensing dye comprises delivering the first and second waves of excitation light to the oxygen-sensing dye within the optical waveguide, such that the phase-shifted second wave continuously traverses the waveguide and is reflected back through the waveguide by the mirror, wherein the reflected second wave interferes with the delayed first wave to cause an interference pattern within the waveguide that excites the dye molecules to emit the signals at a section of the multiple sections along the waveguide, the method further comprising:
delaying the first wave by one or more additional selected time periods different from the first selected time period;
shifting the phase of the second wave; and
delivering the first wave delayed by the one or more additional selected time periods and the phase-shifted second wave to the oxygen-sensing dye within the optical waveguide, such that the phase-shifted second wave continuously traverses the waveguide and is reflected back through the waveguide by the mirror, wherein the reflected second wave interferes with the first wave delayed by the one or more additional selected time periods to cause one or more additional interference patterns that excite the dye molecules to emit the signals at one or more additional sections of the multiple sections along the waveguide.
